(19) United States
(12) Patent Application Publication
(10) Pub. No.: US 2026/0009784 A1
(43) Pub. Date: Jan. 8, 2026

(54) ELECTROPHORETIC SYSTEM AND METHOD FOR ANALYTE CAPTURE

(71) Applicant: 10x Genomics, Inc., Pleasanton, CA (US)

(72) Inventor: Marlon Stoeckius, Stockholm (SE)

(21) Appl. No.: 19/271,897

(22) Filed: Jul. 17, 2025

Related U.S. Application Data
(63) Continuation of application No. 17/150,997, filed on Jan. 15, 2021, now Pat. No. 12,405,264.
(60) Provisional application No. 62/962,559, filed on Jan. 17, 2020.

(51) Int. Cl. G01N 33/487; B01L 3/00; G01N 27/447

(52) U.S. Cl. CPC G01N 33/48721; B01L 3/5025; G01N 27/44791; B01L 2400/0421

(57) ABSTRACT

An electrophoretic system is provided for analyte capture from a biological sample. The electrophoretic system can be used to permeabilize the sample to allow analytes to be released from the sample. For example, the sample can be contacted with capture probes attached to a substrate, and an electric field created by the electrophoretic system can cause analytes to be released from the cell, and effectively migrate toward and bind to the capture probes attached to the substrate.

### CROSS-REFERENCE TO RELATED APPLICATION

This application is a continuation of U.S. patent application Ser. No. 17/150,997, filed on Jan. 15, 2021, which claims the benefit of U.S. Patent Application Ser. No. 62/962,559, titled ELECTROPHORETIC SYSTEM AND METHOD FOR PREPARING SAMPLE, filed Jan. 17, 2020, the disclosure of which is hereby incorporated by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Various methods have been used to capture analytes from a biological sample for analyzing analyte data in the sample. In some applications, a biological sample can be permeabilized to facilitate transfer of analytes out of the sample, and/or to facilitate transfer of species (such as capture probes) into the sample. If a sample is not permeabilized sufficiently, the amount of analyte captured from the sample may be too low to enable adequate analysis.

### SUMMARY

This document generally relates to electrophoretic apparatuses, systems, and methods for capturing analytes from a sample, such as a biological sample.

Some embodiments described herein include an electrophoretic system for analyte capture from a biological sample, such as a cell or a tissue sample including a cell. The electrophoretic system can be used to permeabilize the sample to allow analytes to be released from the sample (e.g., the cell therein). For example, the sample can be contacted with capture probes attached to a substrate (e.g., a surface of the substrate), and an electric field created by the electrophoretic system can cause analytes to be released from the cell, and effectively migrate toward and bind to the capture probes attached to the substrate. Loss of spatial resolution can occur when analytes migrate from a sample to capture probes (e.g., feature array) and a component of diffusive migration occurs in a transverse (e.g., lateral) direction, approximately parallel to the surface of the substrate on which the sample is mounted. The electrophoretic system described herein can actively direct analytes released from a cell to the capture probes, thereby improving spatial resolution by eliminating or reducing such diffusive migration in the transverse direction.

In some implementations, a substrate (e.g., slide) on which a sample (e.g., cell, tissue, etc.) is placed can include a conductive material and used as an anode in the electrophoretic system described herein. An electrode can be placed to be spaced apart from the substrate, and used as a cathode. A non-conductive spacer can be arranged between the substrate and the electrode. The substrate, the electrode, and the spacer can be at least partially immersed in a buffer. The electrophoretic system can apply a voltage between the electrodes (i.e., the substrate comprising the anode, and the electrode as the cathode) to cause analytes to release from the sample and migrate to capture probes attached to the substrate.

In some implementations, the electrophoretic system described herein can include a variety of substrate cassettes configured for analyzing multiple samples. For example, a substrate cassette can be configured to engage a substrate that has multiple substrate regions. The substrate cassette can include a plurality of apertures corresponding to the substrate regions of the substrate, respectively. The plurality of apertures can be used as a plurality of chambers for receiving buffers, respectively. The substrate can be made of a conductive material and used as a common anode for the plurality of chambers. A cathode can be configured to include a plurality of electrode plates or pins that can extend into the chambers at least partially filled with buffers, respectively. The electrophoretic system can apply a voltage between the substrate (as the anode) and the cathode (including the electrode plates or pins) to cause analytes to release from the samples in each of the substrate regions, and migrate to a capture probe provided at each of the substrate regions of the substrate.

Particular embodiments described herein include an electrophoretic system for migrating analytes in a biological sample. The system includes a substrate, a cathode, a buffer chamber, and a controller. The substrate may include a substrate region configured to place a capture probe thereon. The substrate region may be configured to receive the biological sample containing analytes. The substrate may be configured to be usable as an anode. The cathode may be spaced apart from the substrate. The buffer chamber may be disposed between the substrate and the cathode and configured to contain a buffer. The controller may be configured to generate an electric field between the substrate and the cathode such that the analytes in the biological sample migrate toward the capture probe on the substrate.

In some implementations, the system can optionally include one or more of the following features. The substrate may include a conductive material. The substrate may be coated with a conductive material. The conductive material may include at least one of tin oxide (TO), indium tin oxide (ITO), a transparent conductive oxide (TCO), aluminum doped zinc oxide (AZO), or fluorine doped tin oxide (FTO). The substrate may include an array of substrate regions configured to place capture probes thereon. The capture probe may be immobilized on the substrate region. The system may include a spacer disposed between the substrate and the cathode to define the buffer chamber. The buffer may include a permeabilization reagent. The system may include a power supply, and electrical wires connecting the power supply to the substrate and the cathode. The system may include a substrate cassette configured to hold the substrate and include a plurality of apertures configured to define a plurality of buffer chambers on the substrate.

Particular embodiments described herein include a method for migrating analytes in a biological sample to a substate. The method may include placing the biological sample in contact with a capture probe on a substrate, the biological sample including analytes; arranging a cathode relative to the substrate at a distance; providing a buffer between the cathode and the biological sample on the substrate; and generating an electric field between the cathode and the substrate to cause the analytes to migrate toward the capture probe on the substrate.

In some implementations, the system can optionally include one or more of the following features. The capture probe may be immobilized on the substrate. The substrate may include a conductive material. The substrate may be coated with a conductive material. The conductive material may include at least one of tin oxide (TO), indium tin oxide (ITO), a transparent conductive oxide (TCO), aluminum doped zinc oxide (AZO), or fluorine doped tin oxide (FTO). The substrate may include an array of substrate regions configured to place capture probes thereon. The method may include arranging a spacer between the substrate and the cathode to contain the buffer between the substrate and the cathode. The buffer may include a permeabilization reagent. The method may include connecting electrical wires from a power supply with the substrate and the cathode, respectively.

Particular embodiments described herein include an electrophoretic system. The system may include a substrate, a substrate cassette, a cathode assembly, and a controller. The substrate may include a plurality of substrate regions including capture probes and one or more biological samples containing analytes. The substrate cassette may be configured to hold the substrate and include a plurality of apertures corresponding to the plurality of substrate regions of the substrate. The plurality of apertures may be configured to define a plurality of buffer chambers on the plurality of substrate regions of the substrate. The cathode assembly may include a plurality of electrode plates. The plurality of electrode plates may be configured to position within the plurality of buffer chambers of the substrate cassette. The controller may be configured to generate electric fields between the plurality of substrate regions and the plurality of electrode plates, respectively, such that the analytes in the biological samples migrate toward the capture probes on the substrate.

In some implementations, the system can optionally include one or more of the following features. The biological samples may be placed in contact with the capture probes on the plurality of substrate regions. The plurality of substrate regions may include a plurality of wells recessed on the substrate. The substrate cassette may include a substrate holder and a gasket. The substrate holder may include a substrate mount for securing the substrate. The gasket may include a plurality of gasket apertures configured to align with the plurality of substrate regions when the substrate is secured by the substrate holder. The plurality of apertures may include the plurality of gasket apertures. The substrate holder may include a plurality of holder apertures configured to align with the plurality of gasket apertures when the substrate is secured by the substrate holder. The plurality of apertures may include the plurality of gasket apertures and the plurality of holder apertures. The system may include a substrate cover configured to be arranged on the substrate cassette and include a plurality of cover apertures configured to align with the plurality of apertures of the substrate cassette. The cathode assembly may be mounted to the substrate cover and the plurality of electrode plates of the cathode assembly may be configured to extend through the plurality of cover apertures into the plurality of apertures of the substrate. The substrate may be coated with a conductive material. The conductive material may include at least one of tin oxide (TO), indium tin oxide (ITO), a transparent conductive oxide (TCO), aluminum doped zinc oxide (AZO), or fluorine doped tin oxide (FTO). The buffer may include a permeabilization reagent. The system may include a power supply, and electrical wires connecting the power supply to the cathode and each of the plurality of substrate regions of the substrate.

Particular embodiments described herein include a method for capturing analytes from a biological sample. The method may include placing biological samples in contact with capture probes on a substrate, the biological sample including analytes; arranging a substrate cassette onto the substrate to align a plurality of apertures of the substrate cassette with a plurality of substrate regions of the substrate and define a plurality of buffer chambers on the plurality of substrate regions; supplying buffers in the plurality of buffer chambers; arranging a cathode to place a plurality of electrode plates of the cathode within the plurality of buffer chambers; and generating electric fields between the plurality of substrate regions and the plurality of electrode plates to cause the analytes in the biological samples to migrate toward the capture probes on the substrate.

In some implementations, the system can optionally include one or more of the following features. The capture probes may be immobilized on the plurality of substrate regions. The biological samples may be placed in contact with the capture probes on the plurality of substrate regions. The plurality of substrate regions may include a plurality of wells recessed on the substrate. The substrate cassette may include a substrate holder and a gasket. The substrate holder may include a substrate mount for securing the substrate. The gasket may include a plurality of gasket apertures configured to align with the plurality of substrate regions when the substrate is secured by the substrate holder. The plurality of apertures may include the plurality of gasket apertures. The substrate holder may include a plurality of holder apertures configured to align with the plurality of gasket apertures when the substrate is secured by the substrate holder. The plurality of apertures may include the plurality of gasket apertures and the plurality of holder apertures. The method may include providing a substrate cover including a plurality of cover apertures and mounting the cathode assembly; and placing the substrate cover onto the substrate cassette such that the plurality of cover apertures of the substrate cover is aligned with the plurality of apertures of the substrate cassette, respectively, and such that the plurality of plates of the cathode assembly extends through the plurality of cover apertures into the plurality of apertures of the substrate. The substrate may be coated with a conductive material. The conductive material may include at least one of tin oxide (TO), indium tin oxide (ITO), a transparent conductive oxide (TCO), aluminum doped zinc oxide (AZO), or fluorine doped tin oxide (FTO).

The devices, system, and techniques described herein may provide one or more of the following advantages. Some embodiments described herein include an electrophoretic system configured to provide appropriate permeabilization of a sample and reduce lateral diffusion that may result from incomplete permeabilization of the sample, thereby increasing the amount of analytes captured and available for detection.

Further, the electrophoretic system described herein can eliminate or reduce additional permeabilization processes. The electrophoretic system can achieve a desired level of spatial resolution without other types of permeabilization or with reduced additional permeabilization. For example, the electrophoretic system can eliminate a need of a permeabilization agent. Alternatively, the electrophoretic system can permit for a reduced amount of permeabilization agent to be used to achieve a desired level of spatial resolution. For example, prior to electrophoresis, a sample can be contacted with a permeabilization agent only for a shorter period of time than a time for complete permeabilization of the sample. Such incomplete permeabilization of the sample can be compensated by the electrophoretic system described herein that eliminates or reduces lateral diffusion of migrating analytes (i.e., analyte diffusion in the transverse direction—orthogonal to the normal direction to the surface of the sample).

Moreover, the electrophoretic system described herein can cause analytes to actively migrate to capture probes by electrophoretic transfer, thereby permitting for the spatial location of the analytes captured by the capture probes on a substrate to be more precise and representative of the spatial location of the analytes in the biological sample than when the analytes are migrated to the capture probes under different environments.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 schematically illustrates an example electrophoretic system.

FIG. 2 schematically illustrates an example substrate.

FIGS. 3A-D illustrate example substrates and samples undergoing electrophoretic process.

FIG. 4 is a flowchart of an example process for preparing a sample.

FIG. 5 schematically illustrates an example electrophoretic system.

FIG. 6 schematically illustrates an example substrate.

FIG. 7 shows an example substrate holder with a substrate in an assembled state.

FIG. 8 shows a bottom surface of an example substrate holder including a gasket and receiving a substrate.

FIGS. 9A-C show A) a top view of an example substrate holder in an open position, B) a side view of a latitudinal side of the substrate holder and C) is a cross sectional view of the substrate holder.

FIGS. 10A and B illustrate an example placement of a substrate into the substrate holder.

FIG. 11 schematically illustrates an example configuration of a substrate region and a sample undergoing electrophoretic process in each buffer chamber.

FIG. 12 is a flowchart of an example process for preparing a sample.

FIG. 13 schematically illustrates an example system for preparing a sample.

FIG. 14 illustrates another example substrate.

FIGS. 15A-C are A) a perspective view of an example device including a plate and a substrate holder for heating a substrate, B) top perspective view of the plate, and C) a bottom perspective view of the plate.

FIG. 16 is an exploded view of the substrate holder of FIG. 15.

FIGS. 17A-B are A) a top perspective view of a bottom member of the substrate, and B) a bottom perspective view of the bottom member of FIG. 16.

FIG. 18 is a perspective view of the bottom member of FIG. 17A coupled to the plate of FIG. 15.

FIG. 19 is a perspective view of the bottom member of FIG. 17A coupled to a second plate embodiment.

FIGS. 20A-B are A) a front perspective view of a fastener for use with the bottom member of FIG. 17 and B) a back perspective view of the fastener.

FIG. 21 is a perspective view of a gasket for use with the substrate holder of FIG. 16.

FIGS. 22A-B are a A) top perspective view of a top member of the substrate holder, and B) a bottom perspective view of the top member of FIG. 16.

FIGS. 23A-C are A) a perspective view of an example substrate holder, B) an exploded view of the substrate holder, and C) a partial, perspective view of the substrate holder.

FIGS. 24A-C are A) a top perspective view of the substrate, B) a bottom perspective view of the substrate, and C) a side perspective view of the substrate holder of FIG. 23A.

FIG. 25 is a perspective view of an example gasket.

### DETAILED DESCRIPTION

In general, the present disclosure provides electrophoretic apparatuses, systems, and methods for preparing a sample for a spatial analysis described herein.

Some embodiments include an electrophoretic system for preparing a biological sample, such as a cell or a tissue sample including a cell. The electrophoretic system can be used to permeabilize the sample to allow analytes to be released from the sample (e.g., the cell therein). For example, the sample can be contacted with capture probes attached to a substrate (e.g., a surface of the substrate), and an electric field created by the electrophoretic system can cause analytes to be released from the cell, and effectively migrate toward and bind to the capture probes attached to the substrate. The electrophoretic system described herein can actively direct analytes released from a cell to the capture probes, thereby improving spatial resolution by eliminating or reducing diffusive migration.

In some implementations, a substrate on which a sample is placed can include a conductive material that can be used as an anode in the electrophoretic system described herein. An electrode can be placed in an arrangement in the system such that the electrode is spaced apart from the substrate. The electrode can be used as a cathode in the system. A non-conductive spacer can be arranged between the substrate and the electrode. In some embodiments, one or more conductive materials, electrodes, and/or non-conductive spacers can be used in the system described herein. The substrate, the electrode, and the spacer can be at least partially immersed in a buffer. In some embodiments, the substrate, the electrode, the spacer, or any combination thereof, can be fully immersed in a buffer. The electrophoretic system can apply a voltage between the electrodes (i.e., the substrate as the anode, and the electrode as the cathode) to cause analytes to release from the sample and migrate to capture probes attached to the substrate.

In some implementations, a variety of substrate cassettes for analyzing multiple samples can be used with the electrophoresis system provided herein. For example, a substrate cassette can be configured to engage a substrate that has multiple substrate regions. The substrate cassette can include a plurality of apertures corresponding to the substrate regions of the substrate, respectively. The plurality of apertures can be used as a plurality of chambers for receiving buffers, respectively. The substrate can be made of a conductive material and used as a common anode for the plurality of chambers. A cathode can be configured to include a plurality of electrode plates that can extend into the chambers at least partially filled with buffers, respectively. The electrophoretic system can apply a voltage between the substrate (as the anode) and the cathode (including the electrode plates) to cause analytes to release from the samples in each of the substrate regions, and migrate to a capture probe provided at each of the substrate regions of the substrate.

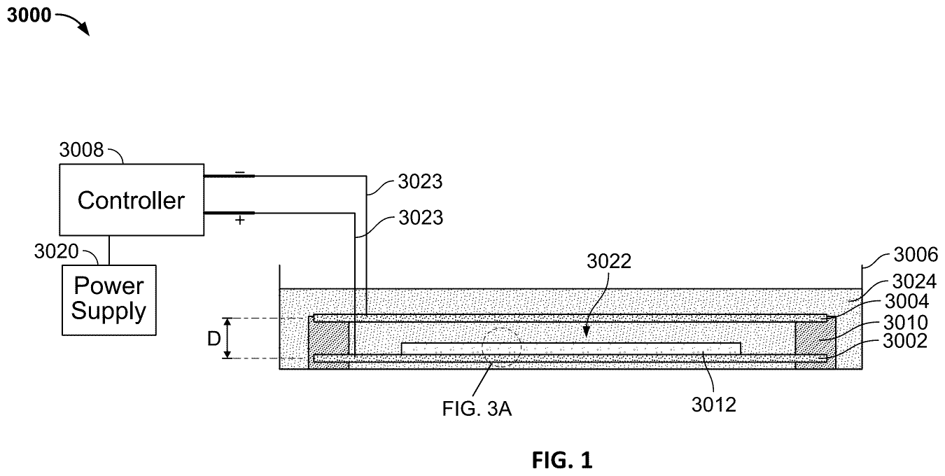

Referring to FIGS. 1-4, an example electrophoretic system 3000 is described for preparing a sample. FIG. 1 schematically illustrates an example configuration of the electrophoretic system 3000. In some implementations, the electrophoretic system 3000 can be used to provide electrophoretic permeabilization of a sample, and/or actively cause analytes in the sample to migrate to capture probes on a substrate. In some embodiments, the electrophoretic system 3000 can be used to enhance electrophoretic permeabilization of a sample by actively directing analytes in the sample with desired directionality. For example, electrophoretic permeabilization by the system 3000 can result in higher analyte capture events (by, e.g., driving more analytes to the capture probes) and better spatial fidelity of captured analytes (e.g., on a feature array) than random diffusion onto matched substrates without the application of an electric (e.g., increases resolution of spatial analyte detection).

In some implementations, the electrophoretic system 3000 can include a substrate 3002 as a first electrode, a second electrode 3004, an electrophoretic container 3006, and a control system 3008. The system 3000 can further include a spacer 3010.

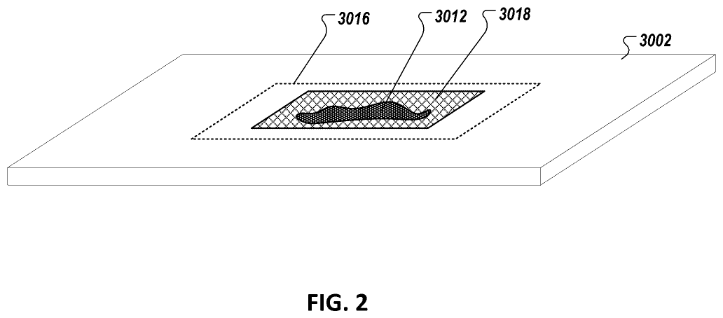

Referring to FIG. 2, the substrate 3002 is configured to receive a sample 3012 that contains analytes. The sample 3012 includes a biological sample, such as a cell or a tissue including a cell. The substrate 3002 can include a substrate region 3016 for receiving the sample 3012 thereon. In some implementations, the substrate 3002 can place a plurality of capture probes 3018 on the substrate region 3016. The capture probes 3018 can be placed on the substrate region 3016 in various manners, such as a variety of ways generally described herein. For example, the capture probes 3018 can be directly attached to a feature that is on an array. Alternatively or in addition, the capture probes 3018 can be immobilized on the substrate region 3016 of the substrate 3002. The sample 3012 can be prepared on the substrate 3002 in various ways generally described herein.

In some implementations, the substrate 3002 is configured to be used as a first electrode in the electrophoretic system 3000. For example, the substrate 3002 can be used as an anode. In another example, the substrate 3002 can be used as a cathode.

The substrate 3002 can be configured as a conductive substrate described generally herein. For example, the substrate 3002 can include one or more conductive materials that permit for the substrate 3002 to function as an electrode (e.g., the anode). Examples of such a conductive material include tin oxide (TO), indium tin oxide (ITO), a transparent conductive oxide (TCO), aluminum doped zinc oxide (AZO), fluorine doped tin oxide (FTO), and any combination thereof. Alternatively or in addition, other materials may be used to provide desired conductivity to the substrate 3002. In some implementations, the substrate 3002 can be coated with the conductive material. For example, the substrate 3002 can include a conductive coating on the surface thereof, and the sample 3012 is provided on the coating of the substrate 3002.

Although the substrate 3002 is illustrated to include a single substrate region 3016 in FIG. 2, other implementations of the substrate 3002 can include a plurality of substrate regions that are configured to place capture probes and/or samples thereon, respectively.

In the illustrated example of FIG. 1, the substrate 3002 is used as the anode, and the second electrode 3004 is configured as a cathode. In this example, therefore, the second electrode 3004 can also be referred to as the cathode 3004. In some implementations, the cathode 3004 can include a conductive plate, one or more pins, or other suitable configurations. In other implementations, the cathode 3004 can include a conductive substrate that is similar to the substrate 3002. In this configuration, in some implementations, the cathode 3004 does not include capture probes, agents, solutions, or other substances or materials that may interact with the sample placed on the substrate 3002.

The substrate 3002 and the cathode 3004 can be arranged within the electrophoretic container 3006. The electrophoretic container 3006 can provide a buffer chamber 3022 between the substrate 3002 and the cathode 3004. The buffer chamber 3022 is configured to contain a buffer 3024. In some implementations, the substrate 3002 and the cathode 3004 can be fully immersed into the buffer 3024. In alternative implementations, either or both of the substrate 3002 and the cathode 3004 can be partially inserted into the buffer 3024 contained in the electrophoretic container 3006.

The buffer 3024 can be of various types. In some implementations, the buffer 3024 includes a permeabilization reagent. In some implementations, the buffer 3024 does not include a permeabilization reagent. The buffer 3024 is contained in the buffer chamber 3022 throughout the electrophoretic process.

The spacer 3010 can be disposed between the substrate 3002 and the cathode 3004 to space them apart at a distance D. The spacer 3010 is made of non-conductive material, such as plastic, glass, porcelain, rubber, etc. The distance D can be determined to provide a desired level of spatial resolution based on several factors, such as the strength and/or duration of electric field generated between the substrate 3002 and the cathode 3004, and other parameters described herein. The spacer 3010 can define at least part of the buffer chamber 3022 between the substrate 3002 and the cathode 3004.

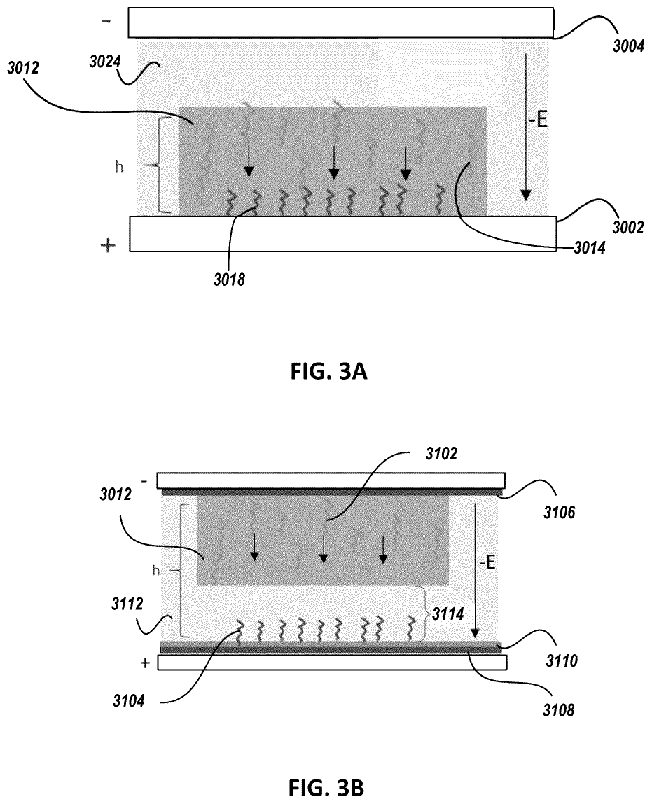

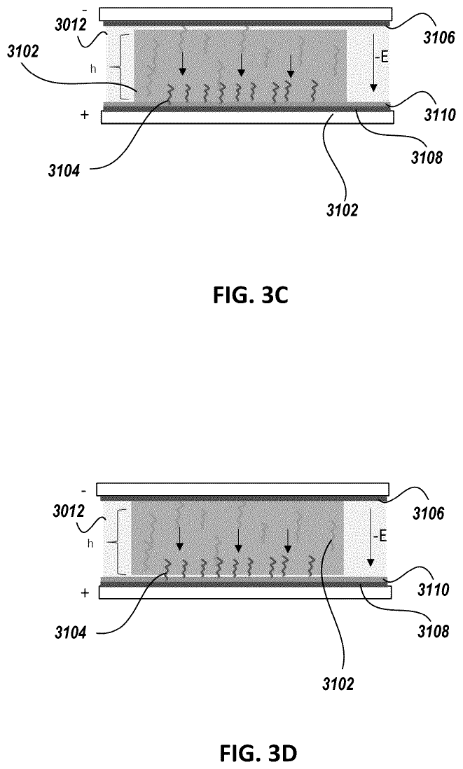

The controller 3008 operates to generate an electric field (−E) between the substrate 3002 and the cathode 3004. As illustrated in FIG. 3, the analytes 3014 in the sample 3012 can migrate toward the capture probes 3018 under the electric field (−E). The controller 3008 can operate to apply a voltage between the substrate 3002 and the cathode 3004 using a power supply 3020. The power supply 3020 can include a high voltage power supply. The controller 3008 can be electrically connected to the substrate 3002 and the cathode 3004 using electrical wires 3023.

FIGS. 3A-D illustrate examples of configurations of a substrate and a sample undergoing electrophoretic process using the electrophoretic system 3000. The application of electric field (−E) causes the analytes 3014 to move towards the capture probes 3018 in the direction of the arrow shown. In some implementations, the analytes 3014 include a protein or a nucleic acid. In some embodiments, the analytes 3014 are negatively charged proteins or nucleic acids. In some embodiments, the analytes 3014 include a positively charged protein or a nucleic acid. In some embodiments, the analytes 3014 includes a negatively charged transcript. For example, the analytes 3014 include a polyA transcript. In some embodiments, the capture probes 3018 are attached to the substrate 3002. In some embodiments, the capture probes 3018 can be attached on a feature of an array array. In some embodiments, the analytes 3014 move towards the capture probes 3018 for a distance (h). In some embodiments, the buffer 3024 (e.g., including a permeabilization reagent) can be in contact with the sample 3012, the substrate 3002, the cathodes 3004, or any combination thereof. The buffer 3024 can include any of the permeabilization reagents disclosed above including but not limited to a permeabilization reagent, a permeabilization buffer, a permeabilization enzyme, a buffer without a permeabilization reagent, a permeabilization gel, and a permeabilization solution.

FIG. 3B shows another example configuration in which the sample 3012 is on a first substrate 3106, and there is a gap 3114 in between the sample 3012 and a coating 3110 on the surface of a second substrate 3108. The coating 3110 can be a conductive coating as described herein. For this embodiment, applied electrophoretic charge causes the analytes 3102 to migrate from the biological sample 3012 on the first substrate, through the buffer 3112 and across the gap 3114 to the capture probes 3104 disposed on the second substrate 3108. FIG. 3C shows another example configuration in which sample 3012 is on coating 3110 of the second substrate 3108, but there is no gap present in between the sample 3012 and the first substrate 3106. FIG. 3D shows another example configuration similar to that of FIG. 3B in which sample 3012 is on the first substrate 3106, but there is no or minimal gap present between the sample 3012 and the second substrate 3108 upon which is located the capture probes 3104.

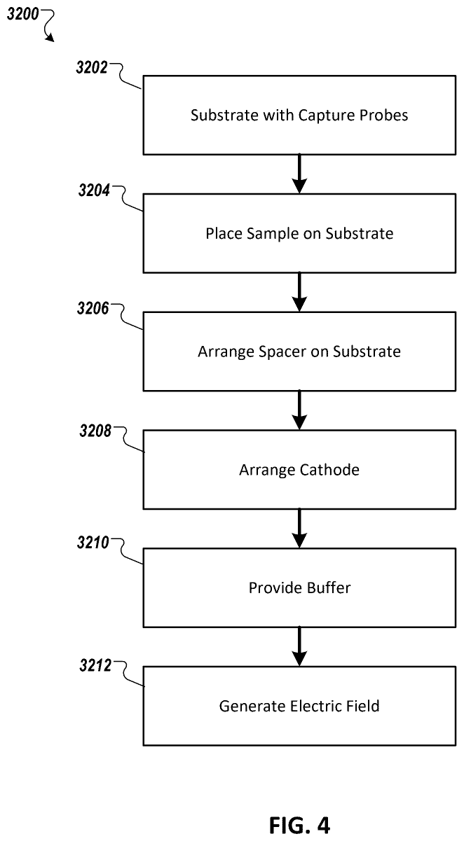

FIG. 4 is a flowchart of an example process 3200 for preparing a sample for use in the electrophoretic systems described herein. In some implementations, the process 3200 includes providing a substrate including capture probes (3202), and placing a sample in contact with the the substrate (3204). The capture probes can be attached to the substrate in various ways generally described herein. The sample can be placed on the substrate in various ways generally described herein. As described herein, the substrate can be configured and used as an electrophoretic electrode (also referred to herein as a first electrode). In some implementations, the substrate can be configured as a conductive substrate as described herein, such as by including a conductive material in the substrate or providing a conductive coating on an upper or lower surface of the substrate. In some implementations, the substrate can be used as an anode. In alternative implementations, the substrate can be used as a cathode.

The process 3200 can further include arranging a spacer on or above the substrate (3206). When a second electrode is arranged as described below, the spacer can be arranged between the second electrode and the first electrode (e.g., the substrate). As described herein, the spacer can be made of a non-conductive material and used to provide a buffer chamber between the first and second electrodes.

The process 3200 can include arranging a second electrode relative to the first electrode (e.g., the substrate) at a distance (3208). The second electrode can be used as a cathode when the substrate is used as an anode. Alternatively, the second electrode can be used as an anode when the substrate is used as a cathode. The second electrode can be made of various configurations. For example, the second electrode can include a conductive plate. Alternatively, the second electrode can be configured as a conductive substrate that is configured similarly to the first electrode (e.g., the substrate).

The process 3200 can include providing a buffer between the first electrode and the second electrode (3210). The buffer can be contained in the buffer chamber that is provided by the spacer and used to at least partially immerse the first electrode (e.g., the substrate), the second electrode, or both. In some implementations, the buffer can include a permeabilization reagent. Other buffers as described generally herein can be used in other implementations. In some embodiments, both electrodes are immersed in a buffer.

The process 3200 can include generating an electric field between the first electrode (e.g., the substrate) and the second electrode (3212). Under the electric field, analytes included in the sample can migrate toward the capture probe on the substrate, wherein the analytes can hybridize to the captures probes. In some implementations, the electric field is generated by applying a voltage between the first and second electrodes, using a power supply electrically connected to the first and second electrodes. For example, the process 3200 can include connecting electrical wires from the power supply with the first electrode (e.g., the substrate) and the second electrode.

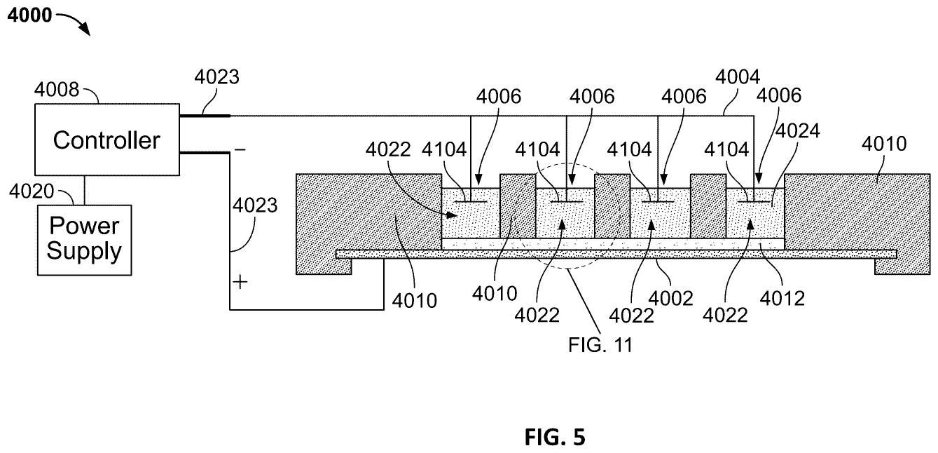

Referring to FIGS. 5-12, another example electrophoretic system 4000 is described. FIG. 5 schematically illustrates an example configuration of the electrophoretic system 4000. In some implementations, the electrophoretic system 4000 can be used to provide electrophoretic permeabilization of a plurality of samples, and/or actively cause analytes in each sample to migrate to capture probes on a substrate. For example, electrophoretic permeabilization by the system 4000 can result in more analytes being captured by capture probes and better spatial fidelity of captured analytes (e.g., on a feature array) than random diffusion onto substrates without the application of an electric field.

In some implementations, the electrophoretic system 4000 can include a substrate 4002 as a first electrode, a second electrode 4004, a control system 4008, and a substrate cassette 4010.

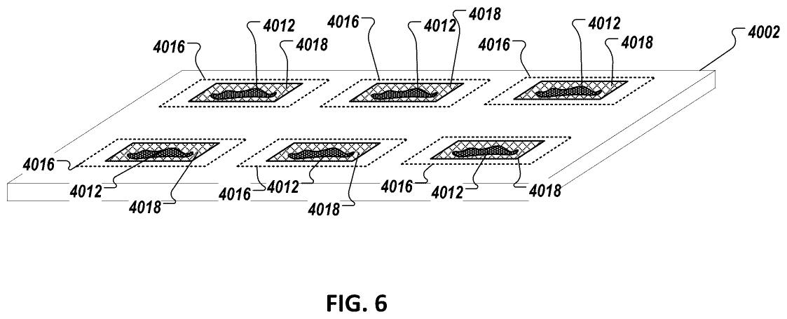

Referring to FIG. 6, the substrate 4002 is configured to receive a plurality of samples 4012 that contain analytes. The samples 4012 can include biological samples 4012, such as a cell or a tissue including a cell. The substrate 4002 can include a plurality of substrate regions 4016 for receiving the sample 4012 thereon. In some implementations, the substrate 4002 a plurality of capture probes 4018 on each substrate region 4016. The capture probes 4018 can be placed on the substrate region 4016 in various manners, such as a variety of ways generally described herein. For example, the capture probes 4018 can be directly attached to a feature on an array. Alternatively or in addition, the capture probes 4018 can be immobilized on the substrate region 4016 of the substrate 4002. The sample 4012 can be prepared on the substrate 4002 in various ways generally described herein.

In some implementations, the substrate 4002 is configured to be used as a first electrode in the electrophoretic system 4000. For example, the substrate 4002 can be used as an anode. In another example, the substrate 4002 can be used as a cathode.

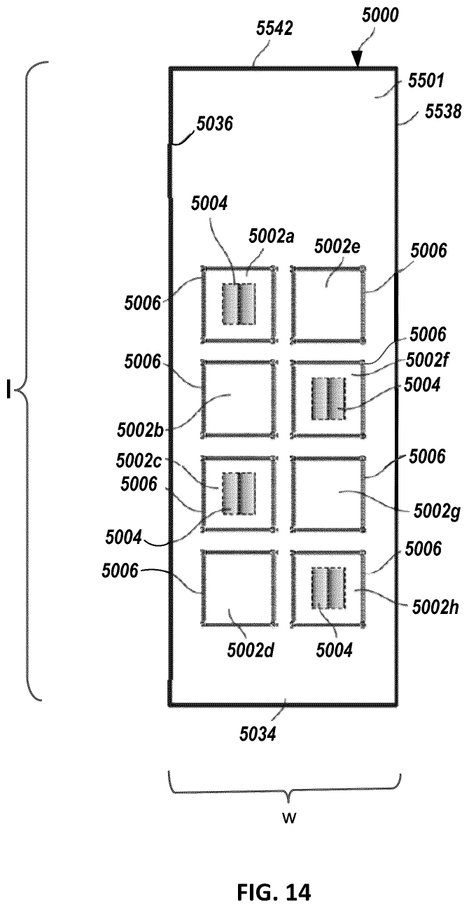

The substrate 4002 can be configured as a conductive substrate as described generally herein. For example, the substrate 4002 can include a conductive material that permits for the substrate 4002 to function as an electrode (e.g., the anode). Examples of such a conductive material include tin oxide (TO), indium tin oxide (ITO), a transparent conductive oxide (TCO), aluminum doped zinc oxide (AZO), fluorine doped tin oxide (FTO), and any combination thereof. Alternatively or in addition, other materials may be used to provide conductivity to the substrate 4002. In some implementations, the substrate 4002 can be coated with the conductive material. For example, the substrate 4002 can include a conductive coating on the surface thereof, and the sample 4012 is provided on the coating of the substrate 4002. Other examples of the substrate 4002 are further described below, for example with reference to FIG. 14.

Referring back to FIG. 5, the substrate cassette 4010 is configured to accommodate the substrate 4002. For example, the substrate cassette 4010 can be configured to immovably mount and hold the substrate 4002. The substrate cassette 4010 can include a plurality of apertures 4006. The plurality of apertures 4006 can be positioned in the substrate cassette 4110 so as to correspond to the plurality of substrate regions 4016 of the substrate 4002 when the substrate cassette 4010 mounts the substrate 4002. The plurality of apertures 4006 can define a plurality of buffer chambers 4022 on the plurality of substrate regions 4106 of the substrate 4002, respectively. The substrate cassette 4010 can be made of non-conductive material, such as plastic, glass, porcelain, rubber, etc.

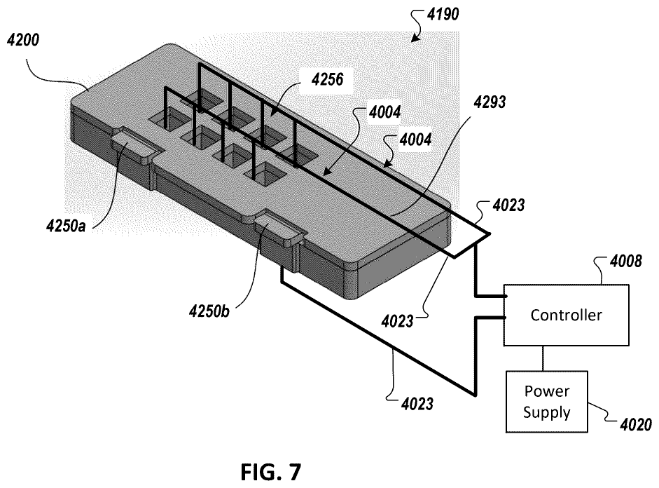

Referring to FIGS. 7-10, an example substrate cassette 4190 is illustrated. The substrate cassette 4190 can be used to implement the substrate cassette 4010 in FIG. 5. The substrate cassette 4190 can include a substrate holder 4200 configured to hold a substrate.

FIG. 7 shows an example substrate holder 4200 with a substrate in an assembled state. This embodiment of the shown substrate holder 4200 can advantageously provide a single-piece component that can be arranged in an open configuration or closed configuration, when desired. In particular, FIG. 7 shows a top surface 4293 of the substrate holder 4200 in a closed position. The substrate holder 4200 includes a plurality of apertures 4256, which can be used to implement the plurality of apertures 4006 of the substrate cassette 4010 of FIG. 5.

The substrate holder 4200 can include a substrate loading mechanism for loading and holding the substrate. For example, the substrate loading mechanism can include a first tab 4250a and a second tab 4250b. In some embodiments, any type of fastener or engagement feature that allows releasable engagement can be used instead of the first and second tabs 4250a and 4250b, such as, for example, screws and press fit type connectors. In some embodiments, the substrate holder 4200 includes 5 tabs or less (e.g., 4 tabs or less, 3 tabs or less, 2 tabs or less, or 1 tab). In some embodiments, the substrate holder 4200 is a single molded unit. Any suitable plastic or polymer can be used as a suitable molding material.

In some embodiments, the substrate holder 4200 includes a substrate mount that has a first surface and a second surface, where the second surface of the substrate mount is configured to mount a substrate for receiving a sample. The substrate holder can include a first portion configured to receive a gasket. The first portion can include a plurality of ribs extending from a surface of the substrate holder. The substrate holder can include a second portion configured to receive a substrate. The first and second portions can be coupled together by a hinge. The first portion can be configured to fold over the second portion to secure the substrate between the first and second portions. In some embodiments, the substrate is a glass slide. In some embodiments, the substrate holder comprises a gasket disposed between the first portion and the second portion of the substrate holder. In some embodiments, the first portion of the substrate holder includes a releasable engagement mechanism configured to secure the first portion to the second portion when the substrate holder is in the closed state. In some embodiments, the first surface of the substrate engages with at least one of the plurality of ribs extending from a surface of the substrate holder. In some embodiments, the second portion defines a recessed cavity formed in the substrate holder configured to receive the substrate. In some embodiments, the second portion can define a cavity configured to receive the substrate.

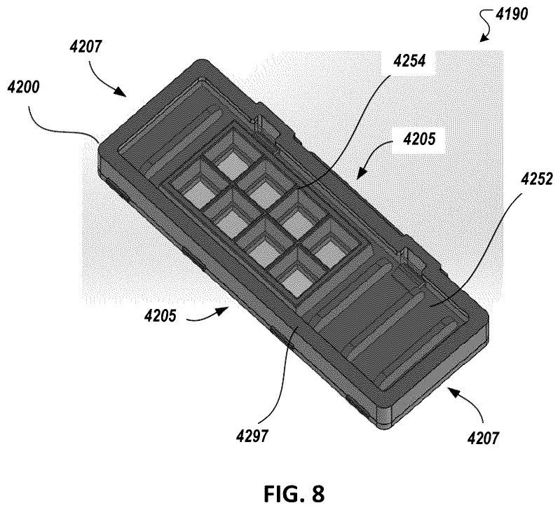

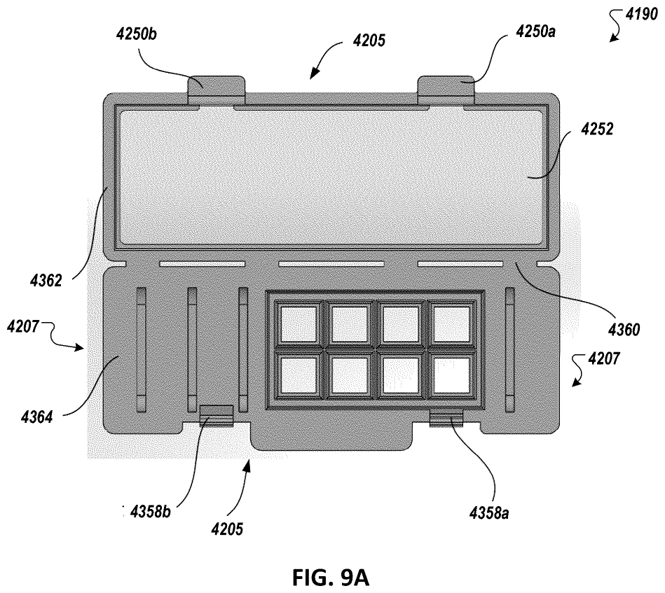

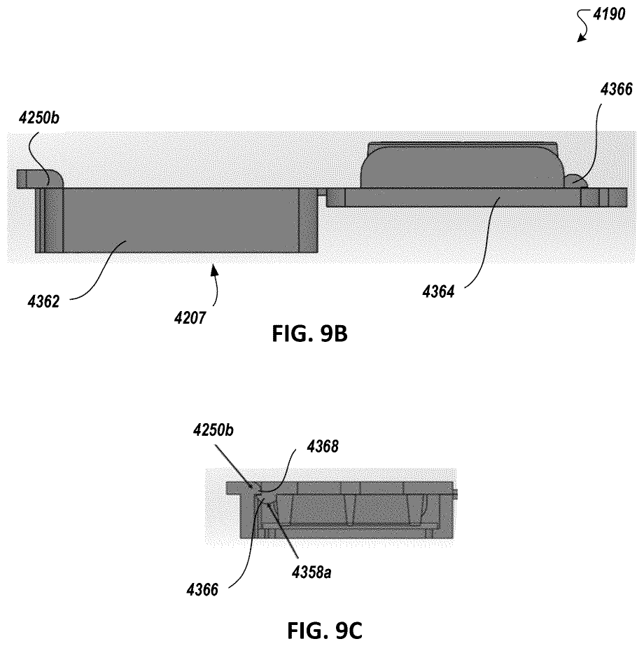

FIG. 8 shows the bottom surface 4297 of the top component of the substrate holder 4200 of the substrate cassette 4190, which includes a gasket 4254 and receiving a slide 4252 (e.g., the substrate). The substrate holder 4200 has longitudinal sides 4205 and latitudinal sides 4207. First and second tabs 4250a and 4250b (FIG. 9), respectively, can protrude from a longitudinal side 4205 of the substrate holder 4200. In some embodiments, the substrate holder 4200 is a single molded unit that includes the gasket 4254. That is, the substrate holder 4200 and the gasket 4254 are one part. In some embodiments, the substrate holder 4200 is overmolded with the gasket 4254. For example, the substrate holder 4200 is a first injection molded plastic part with a second part (e.g., a pliable material) molded onto it to create the gasket 4254. In some embodiments, the pliable material is an elastomer. In some embodiments, the pliable material is silicone rubber. In some embodiments, the gasket 4254 is a separate part that is not molded with the substrate holder 4200.

FIG. 9A shows a top view of the substrate holder 4200 of the substrate cassette 4190 in an open position. The opening and closing mechanism of the substrate holder 4200 is a hinged mechanism. A bottom component 4362 of the substrate holder 4200 can be hinged to a top component 4364 of the substrate holder 4200 via a hinge 4360. In some embodiments, the hinge 4360 can be a living hinge. In some embodiments, the substrate holder 4200 includes 10 hinges or less (e.g., 9 hinges or less, 8 hinges or less, 7 hinges or less, 6 hinges or less, 5 hinges or less, 4 hinges or less, 3 hinges or less, 2 hinges or less, or 1 hinge). Non-limiting examples of hinges that the substrate holder 4200 can include, include a straight or flat living hinge, a butterfly living hinge, a child safe hinge, a double living hinge, and a triple living hinge.

The substrate holder 4200 further includes one or more engagement features, such as a first notch 4358a and a second notch 4358b. The first and second notches 4358a and 4358b can engage the first and second tabs 4250a and 4250b, respectively, when pressed together. The first and second notches 4358a and 4358b can protrude from a longitudinal side 4205 of the top component 4364 of the substrate holder 4200. In some embodiments, the substrate holder 4200 includes three, four, five, six, seven, eight, nine, ten or more notches. In some embodiments, the notches protrude from a latitudinal side 4207 of the substrate holder 4200. In some embodiments, the first and second notches 4358a and 4358b are rigid and do not flex when engaging the first and second tabs 4250a and 4250b, respectively. In some embodiments, the first and second notches 4358a and 4358b, respectively, can be flexible.

FIG. 9B shows a side view of a latitudinal side 4207 of the substrate cassette 4190. The first and second notches 4358a and 4358b can project upward and include a notch ledge 4366 that engages a tab ledge 4368, as shown in FIG. 9C. Alternatively, in some embodiments, the substrate holder 4200 includes a snap fit locking mechanism for releasably receiving and releasably securing the slide 4252. Non-limiting examples of other types of fasteners to be used in locking mechanisms of the substrate holder 4200 include a catch, a projection, a male connector, and a female connector.

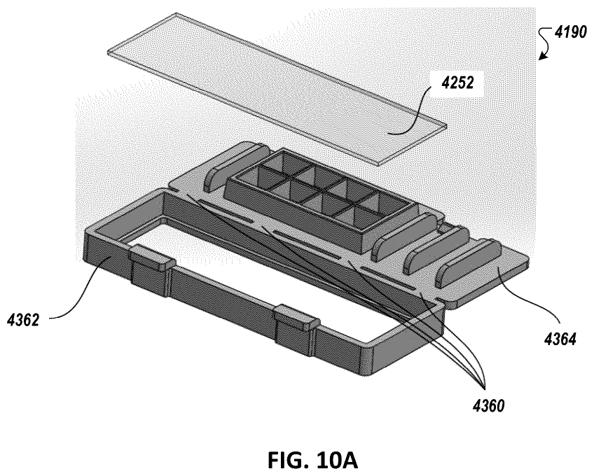

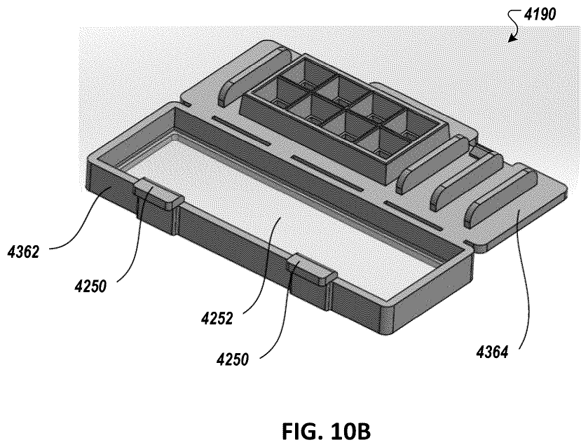

FIGS. 10A and B illustrate the placement of the slide 4252 into the substrate holder 4200 of the substrate cassette 4190. In some embodiments, the slide 4252 can be “loaded” or placed onto an inner rim or an inner edge of the bottom component 4362 of substrate holder 4200. Once loaded, the top component 4364 is closed by pressing the first notch 4358a and the second notch 4358b against the first and second tabs 4250a and 4250b, respectively, thereby forming a tight seal with the slide 4252. In some embodiments, the slide does not have to be tilted under tabs 4250 or any other tabs. In some embodiments, the substrate holder 4200 includes one or more tabs to help load the slide onto the inner rim or inner edge of the bottom component 4362 of substrate holder 4200.

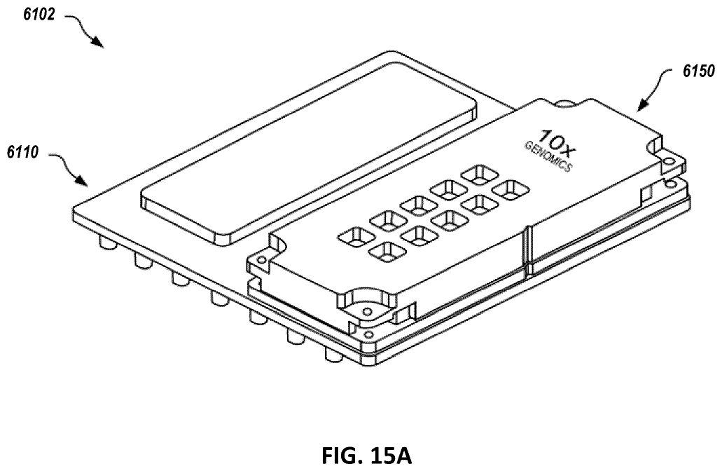

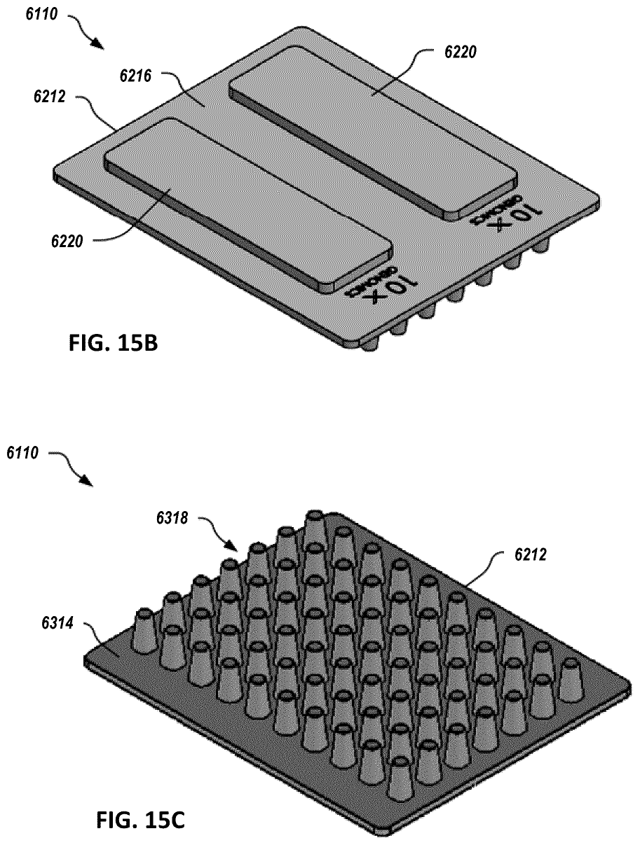

A variety of other configurations of substrate holder can be used to implement the substrate cassette 4010 in FIG. 5. Examples of such other configurations are further illustrated below, for example with reference to FIG. 15.

Referring back to FIG. 5, the substrate 4002 is used as the anode, and the second electrode 4004 is configured as a cathode. In this example, therefore, the second electrode 4004 can also be referred to as the cathode 4004. In some implementations, the cathode 4004 can include a conductive plate, one or more pins, or other suitable configurations. As illustrated, for example, the cathode 4004 can include a plurality of electrode plates 4104. The electrode plates 4104 are configured to be positioned within the plurality of buffer chambers 4022 of the substrate cassette 4010, respectively.

The samples 4012 on the substrate regions 4016 of the substrate 4002, respectively, can be arranged within the buffer chambers 4022 that are defined by the apertures 4006 of the substrate cassette 4010. The buffer chambers 4022 can contain buffers 4024 therein, so that the samples 4012 are fully immersed into the buffers 4024 in the buffer chambers 4022, respectively. Further, as illustrated in FIGS. 5 and 7, the electrode plates 4104 of the cathode 4004 can be arranged within the buffer chambers 4022, respectively. Each of the electrode plates 4104 can be immersed into a buffer 4024 contained in the buffer chamber 4022. The electrode plates 4104 are arranged to be spaced apart from the corresponding substrate regions 4016 of the substrate 4002.

The buffer 4024 can be of various types. In some implementations, the buffer 4024 includes a permeabilization reagent. Alternatively or in addition, other type of buffers as generally described herein can be used. The buffer 4024 is contained in each of the buffer chambers 4022 throughout the electrophoretic process.

The controller 4008 operates to generate an electric field (−E) between the substrate (anode) 4002 and the cathode 4004. For example, the controller 4008 operates to generate an electric field between the substrate regions 4016 of the substrate 4002 and the electrode plates 4104 of the cathode 4004 that correspond to the substrate regions 4016, respectively. Thus, the electric field is generated through each of the buffer chambers 4022. In some implementations, the same electric field is generated for all of the buffer chambers 4022. In other implementations, different electric fields are generated for at least two of the buffer chambers 4022.

In some implementations, the controller 4008 can operate to apply a voltage between the entire substrate 4002 and the entire cathode 4004 using a power supply 4020. In other implementations, the controller 4008 can operate to apply voltages between the substrate regions 4016 of the substrate 4002 and the corresponding electrode plates 4104 of the cathode 4004, respectively. The power supply 4020 can include a high voltage power supply. The controller 4008 can be electrically connected to the substrate 4002 and the cathode 4004 using electrical wires 4023.

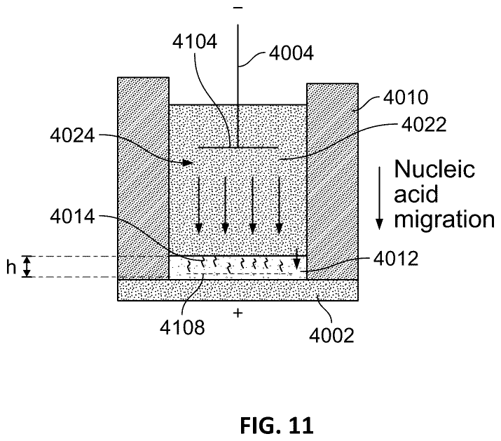

FIG. 11 schematically illustrates an example configuration of a substrate region and a sample undergoing electrophoretic process in each buffer chamber of the electrophoretic system 4000. In each buffer chamber 4022, the analytes 4014 in the sample 4012 can migrate toward the capture probes 4018 under the electric field (−E).

The application of electric field (−E) can cause the analytes 4014 (e.g., negatively charged analytes) to move towards the capture probes 4018 (e.g., positively charged analytes) in the direction of the arrow shown. In some implementations, the analytes 4014 include a protein or a nucleic acid. In some embodiments, the analytes 4014 are negatively charged proteins or nucleic acids. In some embodiments, the analytes 4014 include a positively charged protein or a nucleic acid. In some embodiments, the analytes 4014 includes a negatively charged transcript. For example, the analytes 4014 include a polyA transcript. In some embodiments, the capture probes 4018 are affixed on the substrate 4002 at the substrate regions 4016 of the substrate 4002. In some embodiments, the capture probes 4018 are location at a feature on the array, or can be replaced at a feature on the array. In some embodiments, the analytes 4014 move towards the capture probes 4018 for a distance (h). In some embodiments, the buffer 4024 (e.g., comprising a permeabilization reagent) can be in contact with the sample 4012, the substrate 4002 (e.g., the substrate region 4016 thereof), the cathode 4004 (e.g., the electrode plates 4104 thereof), or any combination thereof. The buffer 4024 can include any of the permeabilization reagents disclosed including but not limited to a permeabilization reagent such as a permeabilization enzyme, a permeabilization buffer, a buffer without a permeabilization reagent, a permeabilization gel, and a permeabilization solution.

In other implementations, the electrophoretic system 4000 can be configured to provide different configurations in the buffer chambers 4024, using, for example, those described in FIG. 3 above.

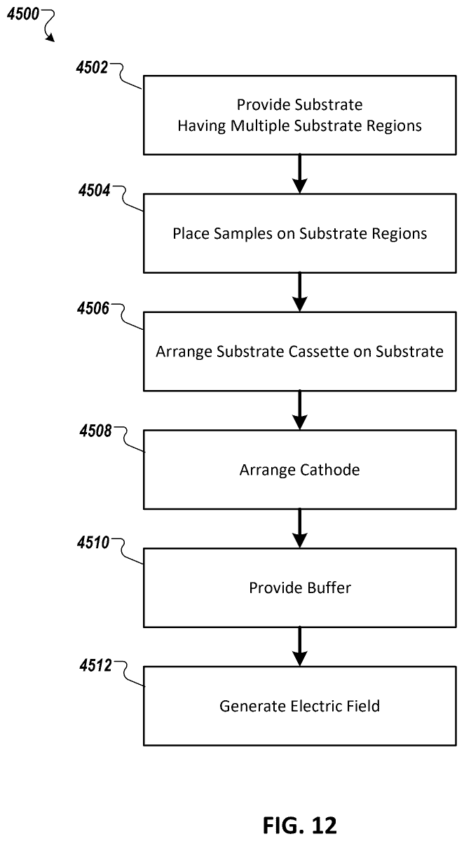

FIG. 12 is a flowchart of an example process 4500 for preparing a sample. In some implementations, the process 4500 includes providing a substrate including a plurality of substrate regions (4502). Each substrate region of the substrate can include a plurality of capture probes. The process 4500 can include placing multiple samples in contact with the capture probes on the substrate regions of the substrate (4504), for example, when there are two substrate regions one sample is contacted to each region, when there are three regions, there are three samples (one on each region), etc. The capture probes can be attached to the substrate regions in various ways described herein. The sample can be placed on the substrate region in various ways described herein.

The substrate can be configured and used as an electrophoretic electrode (also referred to herein as a first electrode). In some implementations, the substrate can be configured as a conductive substrate as described herein, such as by including a conductive material in the substrate or providing a conductive coating on an upper or lower surface of the substrate. In some implementations, the substrate can be used as an anode. In alternative implementations, the substrate can be used as a cathode.

Alternatively, the substrate can be configured and used such that each substrate region operates as an electrophoretic electrode (also referred to herein as a first electrode), while the other portion (e.g., at least a portion around each conductive substrate region) is non-conductive. In some implementations, each substrate region can be configured as a conductive region, such as by including a conductive material in the substrate region or providing a conductive coating on an upper or lower surface of the substrate region. In some implementations, each substrate region can be used as an anode. In alternative implementations, each substrate region can be used as a cathode.

The process 4500 can further include arranging a substrate cassette on or above the substrate (4506). The substrate cassette can be arranged such that a plurality of apertures of the substrate cassette are aligned with the substrate regions of the substrate, respectively, thereby defining a plurality of buffer chambers on the plurality of substrate regions. As described herein, the substrate cassette can be at least partially made of a non-conductive material and used to provide the buffer chambers between the first and second electrodes.

The process 4500 can include arranging a second electrode relative to the first electrode (e.g., the substrate or each substrate region) at a distance (4508). The second electrode can be used as a cathode when the substrate or each substrate region is used as an anode. Alternatively, the second electrode can be used as an anode when the substrate or each substrate region is used as a cathode. The second electrode can be made of various configurations. For example, the second electrode can include a plurality of electrode plates configured to be placed within the buffer chambers, respectively.

The process 4500 can include providing a buffer between the first electrode (e.g., the substrate or each substrate region) and the second electrode (e.g., each electrode plate) (4510). The buffer can be contained in each buffer chamber that is provided by the substrate cassette and used to at least partially immerse the first electrode (e.g., the substrate or each substrate region), the second electrode (e.g., each electrode plate), or both. In some implementations, the buffer can include a permeabilization reagent.

The process 4500 can include generating an electric field between the first electrode (e.g., the substrate or each substrate region) and the second electrode (e.g., each electrode plate) (4512). Under the electric field, analytes included in the sample can migrate toward the capture probes on each substrate region, for example in order to hybridize (e.g., captured) to the capture probe. In some implementations, the electric field is generated by applying a voltage between the first and second electrodes, using a power supply electrically connected to the first and second electrodes. For example, the process 4500 can include connecting electrical wires from the power supply with the first electrode (e.g., the substrate or each substrate region) and the second electrode (e.g., each electrode plate).

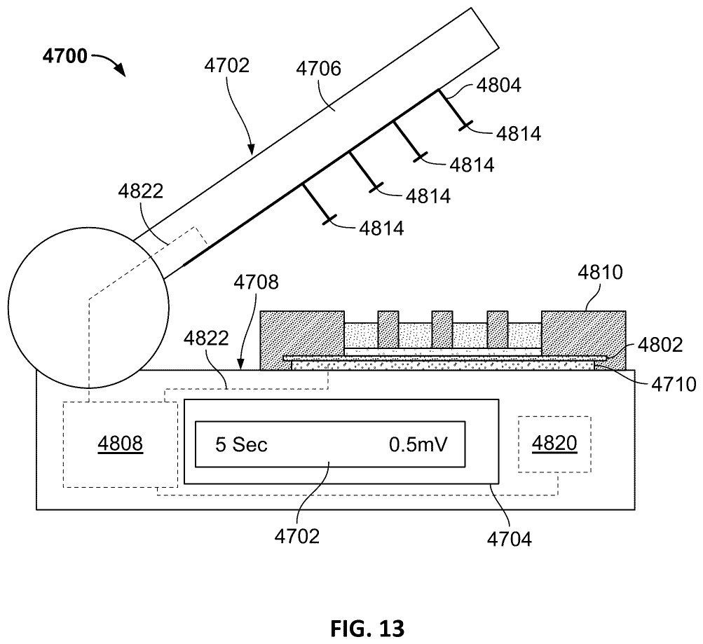

FIG. 13 schematically illustrates an example system 4700 for preparing a sample. The system 4700 can include a sample preparation instrument 4702, a substrate 4802 as a first electrode, a second electrode 4804, a control system 4808, and a substrate cassette 4810.

The sample preparation instrument 4702 is configured to support the substrate cassette 4810 with the substrate 4802, and provide the second electrode 4804 to generate an electric field between the substrate 4802 and the second electrode 4804. In some implementations, the sample preparation instrument 4702 includes a body 4704 and a lid 4706 configured to be place over the body 4704. For example, the lid 4706 is hingedly connected to the body 4704. Alternatively, the lid 4706 can be coupled to the body 4704 in other configurations. In yet alternative implementations, the lid 4706 is configured to be separate from the body 4704, or detachably attached to the body 4704.

In some implementations, the body 4704 provides a supporting surface 4708 configured to support the substrate cassette 4810 including the substrate 4802. The body 4704 further provides an electrical connector 4710 for electrically contacting the substrate 4802 so that the substrate 4802 can be used as an electrode (e.g., an anode) for electrophoresis. The substrate 4802 and the substrate cassette 4810 can be configured identically or similarly to the substrate 4002 and the substrate 4010, respectively. For example, the substrate 4802 includes a plurality of substrate regions that include capture probes configured to place samples thereon. The substrate cassette 4810 includes a plurality of apertures that correspond to the plurality of substrate regions and define a plurality of buffer chambers.

The lid 4706 can be configured to mount the second electrode 4804 (e.g., a cathode). The second electrode 4804 can be configured similarly to the second electrode 4004. For example, the second electrode 4804 can include a plurality of electrode plates 4814 corresponding to the buffer chambers on the substrate 4802. The second electrode 4804 is arranged such that, when the lid 4706 is closed onto the body 4704, or comes close to the body 4704, the electrode plates 4814 of the second electrode 4004 are at least partially inserted into the buffer chambers of the substrate cassette 4810.

The sample preparation instrument 4702 can include the control system 4808 that is identical or similar to the controller 4008. For example, the control system 4808 can be housed in the body 4704. Alternatively, at least part of the control system 4808 can be housed in or mounted to another part of the instrument, such as the lid 4706. Alternatively, the control system 4808 can be at least partially configured as a separate apparatus from the instrument 4702 and electrically connected to the instrument 4702. The control system 4808 can be electrically connected to the substrate 4802 (or each substrate region thereof) and the second electrode 4804 (or each electrode plate thereof) using electrical wires 4822.

The sample preparation instrument 4702 can further include a power supply 4820 configured to apply a voltage between the substrate 4802 (or each substrate region thereof) and the second electrode 4804 (or each electrode plate thereof). The power supply 4820 can be housed in the body 4704. Alternatively, the power supply 4820 can be housed in or mounted to another part of the instrument, such as the lid 4706. Alternatively, the power supply 4820 can be provided separately from the instrument 4702.

In some implementations, the sample preparation instrument 4702 can be configured to automatically start electrophoresis when the lid 4706 is closed over the body 4704, or lowered to a predetermined position (or angle) over the samples, and stop electrophoresis when the lid 4706 returns to be opened or raised from the predetermined position. Alternatively, the sample preparation instrument 4702 can provide a user interface 4720 (e.g., a button, switch, etc.) to receive a manual input of starting or stopping the electrophoretic process. The user interface 4720 can include an output device, such as a display, lamps, etc., configured to output operating parameters of the instrument 4702 (e.g., a voltage being applied, a duration of such application, etc.) or other information associated with the system 4700. The user interface 4720 can further include an input device, such as physical or virtual buttons, switches, keypads, etc., configured to receive a user input of adjusting the operating parameters of the instrument 4702 or other information associated with the system 4700.

Referring to FIG. 14, another example substrate 5000 is described. The substrate 5000 can be used to implement the substrates 4002, and 4802 described herein. The substrate 5000 has a surface 5501 that includes substrate regions 5002a-h. The substrate 5000 can include a first substrate region 5002a, a second substrate region 5002b, a third substrate region 5002c, a fourth substrate region 5002d, a fifth substrate region 5002e, a sixth substrate region 5002f, a seventh substrate region 5002g, and an eighth substrate region 5002h. In some embodiments, substrate 5000 can have less than eight substrate regions or more than eight substrate regions. Each substrate region can be enclosed within a defined perimeter of a frame 5006, for example a frame comprising fiducial markers.

In some implementations, the eight substrate regions 5002a-h can be positioned at the center of the substrate 5000. For example, the eight substrate regions 5002a-h can be centered on the substrate 5000 such that a first distance extending from an outer edge of a frame 5006 of one of the first substrate region 5002a, second substrate region 5002b, third substrate region 5002c, or fourth substrate region 5002d to a longitudinal edge (i.e., along length l) of the substrate 5000 is substantially the same to a second distance extending from an outer edge of a frame 5006 of one of the fifth substrate region 5002e, sixth substrate region 5002f, seventh substrate region 5002g, or eighth substrate region 5002h to a longitudinal edge (i.e., along length l) of substrate 5000. For example, the substrate arrays can be positioned about 28.5 mm from the top edge 5032 of substrate, about 11.25 mm from the bottom edge 5034 of substrate, and about 3.5 mm from the left edge 5036 and right edge 5038 of substrate, when the substrate is oriented vertically as is shown in FIG. 14.

In some embodiments, the eight substrate regions 5002a-h can be centered on the substrate 5000 such that a first distance extending from an outer edge of a frame 5006 of one of the first substrate region 5002a, second substrate region 5002b, third substrate region 5002c, or fourth substrate region 5002d to a latitudinal edge (i.e., along width w) of the substrate 5000 is substantially the same to a second distance extending from an outer edge of a frame 5006 of one of the fifth substrate region 5002e, sixth substrate region 5002f, seventh substrate region 5002g, or eighth substrate region 5002h to a longitudinal edge (i.e., along width w) of the substrate 5000.

In some embodiments, the substrate 5000 can be rectangular in shape. In some embodiments, the substrate 5000 has a length/of about 100 mm to about 10 mm (e.g., 90 mm or less, 85 mm or less, 90 mm or less, 75 mm or less, 70 mm or less, 65 mm or less, 60 mm or less, 55 mm or less, 50 mm or less, 45 mm or less, 40 mm or less, 35 mm or less, 30 mm or less, 25 mm or less, 20 mm or less, 15 mm or less). In some embodiments, the substrate 5000 has a width w of about 100 mm to about 10 mm (e.g., 90 mm or less, 85 mm or less, 90 mm or less, 75 mm or less, 70 mm or less, 65 mm or less, 60 mm or less, 55 mm or less, 50 mm or less, 45 mm or less, 40 mm or less, 35 mm or less, 30 mm or less, 25 mm or less, 20 mm or less, 15 mm or less). In some embodiments, substrates of the disclosure can be square or circular in shape. In some embodiments, substrates of the disclosure can be rectangular, triangular, hexagonal, octagonal, pentagonal, or any other suitable two-dimensional, geometric shape.

In some embodiments, the substrate 5000 includes 1, 2, 3, 4, 5, 6, 7, 8, 9, 10 or more substrate arrays. The substrate 5000 includes four substrate arrays 5004, as shown in FIG. 14. The substrate 5000 can include a substrate array 5004 arranged within the frame 5006. For example, the substrate array 5004 can be positioned at the center of a frame 5006. In some embodiments, the substrate arrays 5004 can be the same (e.g., can have a same pattern). In some embodiments, the substrate arrays 5004 can be the different (e.g., can have a different pattern). The four substrate arrays 5004, shown in FIG. 14, can be repeated and arranged in a checked pattern. That is, substrate array 5004 is positioned at the center of the first substrate region 5002a, the third substrate region 5002c, the sixth substrate region 5002f, and the eighth substrate region 5002h. In some embodiments, the checked pattern is meant to occupy as much of the surface of the substrate to ensure consistency of image acquisition when scanning different regions of the substrate.

In some embodiments, the substrate regions 5002a-h can be arranged vertically in two columns, as shown in FIG. 14. In some embodiments, the substrate regions are arranged on the substrate in one column. In some embodiments, the substrate regions are arranged on the substrate in 2, 3, 4, 5, 6, 7, 8, 9, 10,or more columns. In some embodiments, the substrate arrays 5004 can be positioned adjacent to each other. For example, in some embodiments, the substrate arrays 5004 can be arranged vertically in a column (e.g., positioned on the first four substrate regions 5002a-2402d). Alternatively, in other embodiments, the substrate arrays 5004 can be positioned on the substrate regions 5002a, 5002b, 5002e, and 5002f. In some embodiments, the substrate arrays 5004 can be positioned on the substrate regions 5002e, 5002f, 5002g, and 5002h. In some embodiments, the substrate arrays 5004 can be positioned on the substrate regions 5002c, 5002d, 5002e, and 5002h. In some embodiments, the substrate arrays 5004 can be positioned on the substrate regions 5002e, 5002b, 5002g, and 5002d. In some embodiments, the substrate arrays 5004 are not arranged in a particular pattern. In some embodiments, the substrate arrays 5004 vary individually in size. For example, in some embodiments, a first substrate array may have a greater size than the size of a second substrate array. In some embodiments, a first substrate array may have a smaller size than the size of a second substrate array. In some embodiments, the distance between substrate arrays may vary and be different. For example, in some embodiments, the distance between a first substrate array and a second substrate array may be different than the distance between a third substrate array and a fourth substrate array. In various embodiments, the substrate 5000 can have eight substrate arrays 5004 or less (e.g., 7 substrate arrays or less, 6 substrate arrays or less, 5 substrate arrays or less, 4 substrate arrays or less, 3 substrate arrays or less, 2 substrate arrays or less). In some embodiments, the substrate arrays are arranged on the substrate in one column. In some embodiments, the substrate arrays are arranged on the substrate in 2, 3, 4, 5, 6, 7, 8, 9, 10, or more columns.

Referring to FIGS. 15-22, an example substrate cassette is described. In some implementations, an example system 6102 is configured to heat a substrate and includes a plate 6110 and a substrate holder 6150. The substrate holder 6150 can be used to replace the substrate cassette 4010, 4810 described herein. The plate 6110 can be configured to be received by a heating device (e.g., a thermocycler) and provide heat transfer between the heating device and the substrate holder 6150. The substrate holder 6150 holds one or more substrates (such as one or more microscope slides), and can removably couple to the plate 6110 to facilitate heat transfer from the plate 6110 to the one or more substrates.

Referring to FIGS. 15B and 15C, the plate 6110 can include a platform 6212 with a first surface 6314 and a second surface 6216. The plate 6110 can further include a plurality of members 6318 extending from the first surface 6314. In some embodiments, the plate 6110 can include one or more support members 6220.

The plate 6110 can be generally formed of thermally conductive material to facilitate heat transfer between a heating device and the substrate holder 6150. In some embodiments, the plate 6110 can be made of a metal such as (but not limited to) aluminum and/or stainless steel.

In general, the platform 6212 is configured to be received by a heating device. More specifically, the platform 6212 is generally configured such that thermal transfer occurs between the heating elements of a heating device and the platform 6212. The heat transferred to the platform 6212 is then further transferred to a substrate, as will be discussed in greater detail below.

The plurality of members 6318 can be dimensioned to be received by different regions of a heating device. For example, in some embodiments, the plurality of members 6318 can be dimensioned to be received within individual sample wells of a thermocycler (for example, thermocycler wells that are generally dimensioned to receive small volume test tubes such as 200 μL tubes). In general, the platform 6212 can include any number of members 6318. For example, in some embodiments, the platform 6212 includes between 4 members and 96 members. Typically, heat is more evenly transferred to the platform 6212 when the number of members 6318 is larger, and they are distributed relatively evenly on the surface 6314.

The support member 6220 can extend from the platform 6212 and be configured to couple to the substrate holder 6150. In some embodiments, the support member 6220 can include one or more recesses or protrusions that couple to one or more complementary protrusions or recesses on the substrate holder 6150 to aid in coupling the substrate holder 6150 to the plate 6110. In some embodiments, the support member 6220 can be entirely received by a portion of the substrate holder 6150. In some embodiments, a portion of the support member 6220 can be received by a portion of the substrate holder 6150. In some embodiments, the support member 6220 can be substantially flat on a top face. In some embodiments, the support member 6220 can be sized and positioned such that the plate 6110 can include multiple support members 6220. For example, in some embodiments, the plate 6110 can include two support members 6220.

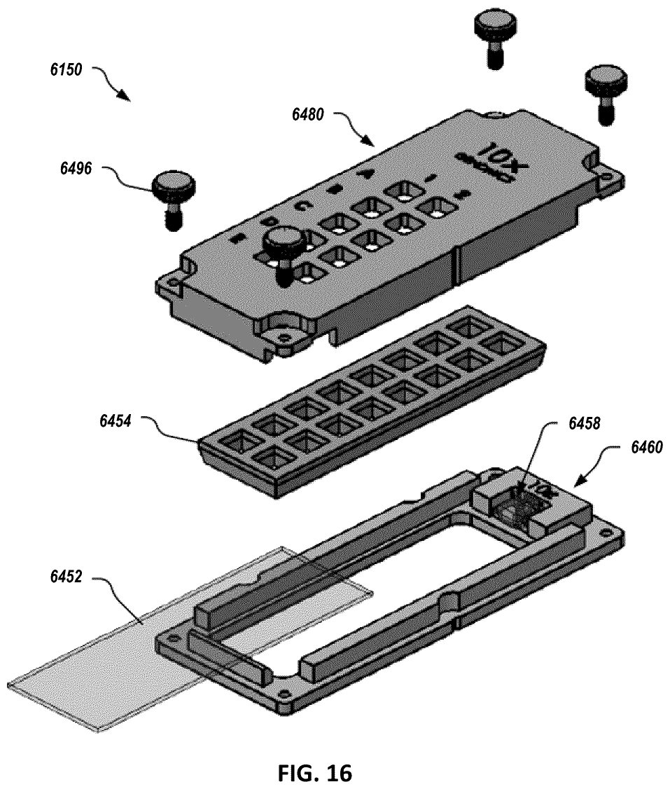

Referring generally to FIGS. 16-22, a substrate holder 6150 can include a bottom member 6460 and a top member 6480. In some embodiments, the substrate holder 6150 can further include a slide 6452. In some embodiments, the substrate holder 6150 can include a gasket 6454 (see also FIG. 21).

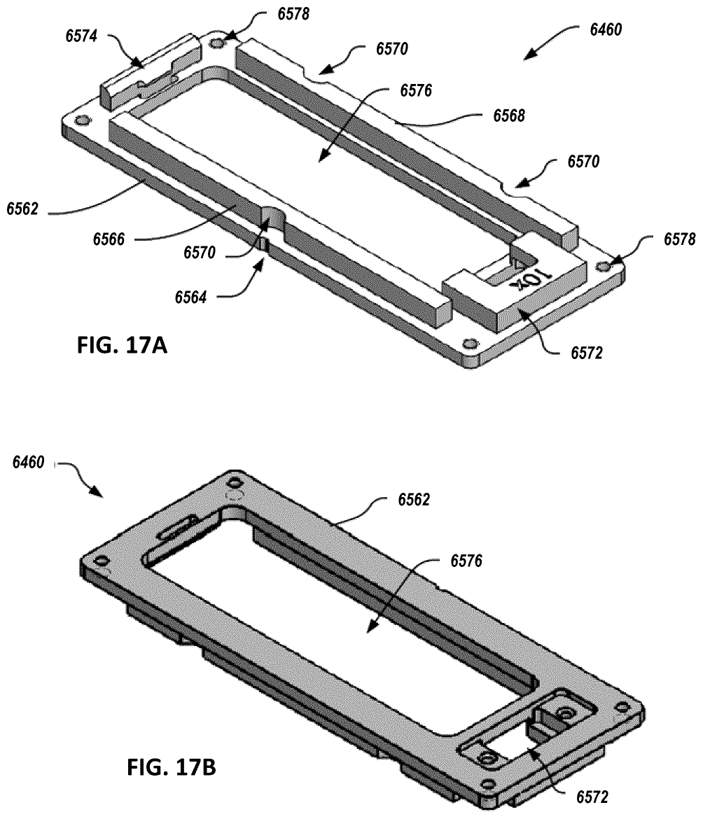

Referring to FIGS. 17A and 17B, the bottom member 6460 can include a base 6562, a first side wall 6566 and a second side wall 6568. The bottom member 6460 can be configured to mount slide 6452.

In some embodiments, the base 6562 can include a marker 6564. The marker 6564 can aid in coupling the bottom member 6460 and the top member 6480 in a certain orientation. For example, in some embodiments, the bottom member 6460 and the top member 6480 may only be able to couple together in a single orientation. In some embodiments, the marker 6564 can be a line, groove, indent, protrusion, symbol, color, etc. to distinguish one side of the base 6562 from another side of the base 6562.

The first side wall 6566 and the second side wall 6568 can be positioned on opposing longitudinal sides of the base 6562. The side walls 6566 and 6568 can extend substantially perpendicular from the base 6562. In some embodiments, the side walls 6566 and 6568 are slightly offset on edges of the base 6562 such that a portion of the base 6562 is exposed on either side of both side walls 6566 and 6568. The side walls 6566 and 6568 can aid in securing the slide 6452 in the bottom member 6460. In some embodiments, when the slide 6452 is secured in the bottom member 6460, the slide 6452 may rest on the base 6562. In some embodiments, the side walls 6566 and 6568 can be configured to engage with the top member 6480. For example, in some embodiments, the side walls 6566 and 6568 can include one or more recesses 6570. In some embodiments, the side wall 6566 can include a single recess 6570, while the side wall 6568 includes multiple recesses (e.g., two recesses). Such a configuration can provide single direction coupling of the bottom member 6460 and the top member 6480 for ease of use.

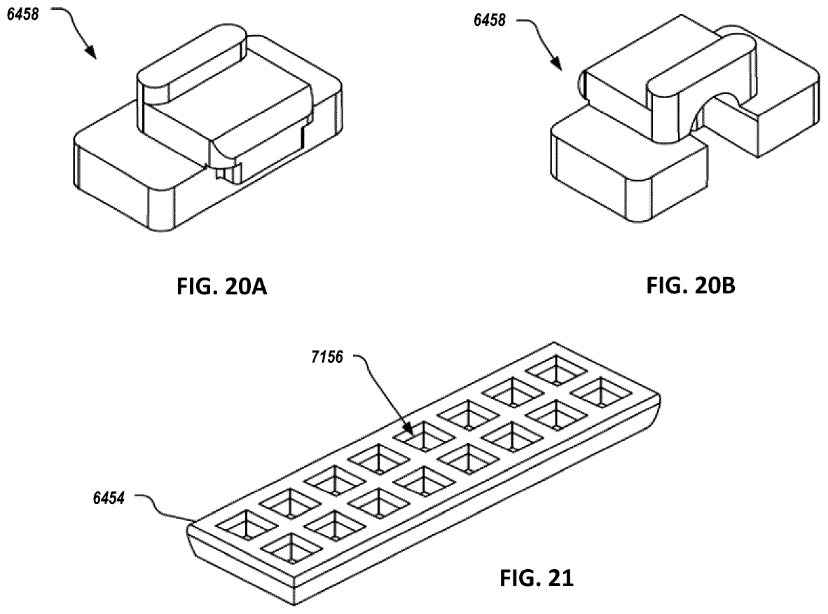

The base 6562 can further include means for mounting the slide 6452. For example, in some embodiments, the base 6562 can include a fastener housing 6572 and an end securing member 6574. The end securing member 6574 can aid in securing the slide 6452 in the bottom member 6460. In some embodiments, the end securing member 6574 can include a ridge to limit movement of the slide 6452 away from the base 6562. The fastener housing 6572 can provide housing for a fastener 6458 (see e.g., FIG. 20). The fastener 6458 can be a clip, ridge, or other means of removably coupling the slide 6452 to the bottom member 6460. In some embodiments, the fastener 6458 can include a spring such that pushing the slide 6452 against the fastener 6458 causes the fastener 6458 to move past the ridge of the end securing member 6574, allowing the slide 6452 to enter the bottom member 6460. Once the slide 6452 is released, the spring can return the fastener 6458 to a more neutral position, causing the slide 6452 to abut the end securing member 6574.

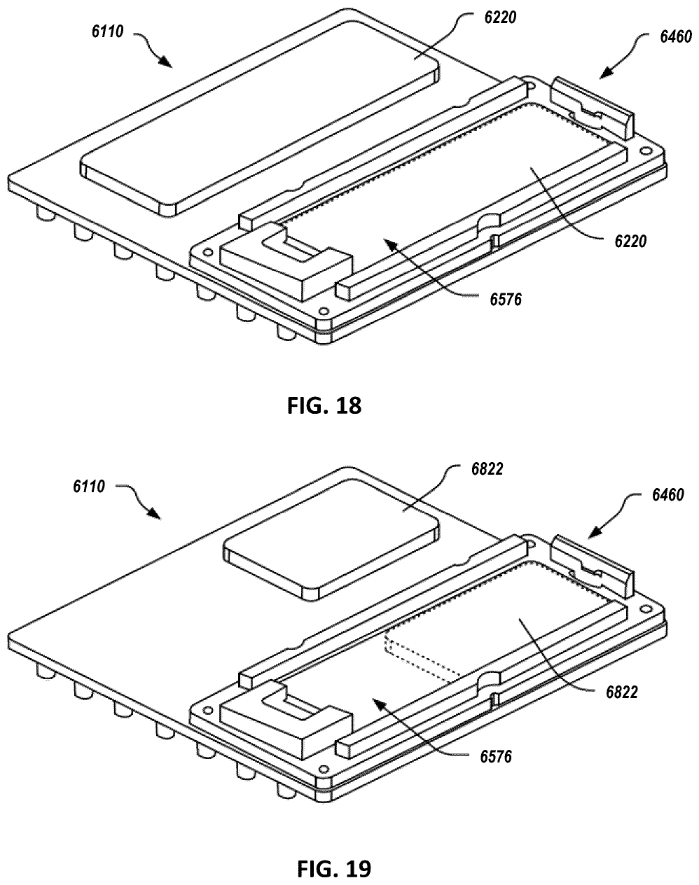

The bottom member 6460 can include means for coupling to the support member 6220 of the plate 6110. For example, in some embodiments, the base 6562 can include an aperture 6576 extending through the base 6562 within the side walls 6566 and 6568. Referring to FIGS. 25 and 26, the bottom member 6460 is shown coupled to the plate 6110. In some embodiments, the aperture 6576 can be sized such that the support member 6220 substantially fills the aperture 6576 (see FIG. 18). In some embodiments, the aperture 6576 can be larger than the support member 6822, such that the support member 6220 only fills a portion of aperture 6576 (see FIG. 19). In some embodiments, the support member 6220, the bottom member 6460 and the aperture 6576 are configured such that the slide 6452 is in close proximity to the support member 6220. In some embodiments, the support member 6220, the bottom member 6460 and the aperture 6576 are configured such that the slide 6452 is in direct contact with the support member 6220. In some embodiments, the support member 6220, the bottom member 6460 and the aperture 6576 are configured such that a portion of a sample region of the slide 6452 is in proximity to the support member 6220. In some embodiments, the portion of the sample region of the slide 6452 is 50% to 100% of the sample region. In some embodiments, the portion of the sample region is at least 60% of the sample region. In some embodiments, the portion of the sample region is at least 75% of the sample region. In some embodiments, the portion of the sample region is at least 80% of the sample region. In some embodiments, the portion of the sample region is at least 85% of the sample region.

Referring back to FIGS. 16-22, the bottom member 6460 can include an engagement mechanism for coupling the bottom member 6460 to the top member 6480. In some embodiments, the engagement mechanism includes screws 6496. Accordingly, the base 6562 can include one or more threaded apertures 6578 configured to receive the screws 6496. The base 6562 can be sized such that the screws 6496 do not protrude underside of the base 6562.

The gasket 6454 can be positioned inside the substrate holder 6150. The gasket 6454 can include a plurality of apertures 7156 (see also FIG. 21) that can create a plurality of wells when the gasket 6454 abuts the slide 6452. In some embodiments, the gasket 6454 can be made of rubber, silicone, or a similar material to create a seal with the slide 6452. In some embodiments, the gasket 6454 can be made of a material that is hydrophobic. Accordingly, different reactions can be conducted in the various wells of the gasket 6454. In some embodiments, the engagement of the bottom member 6460 and the top member 6480 creates ample pressure to maintain division between the wells created by the apertures 7156.

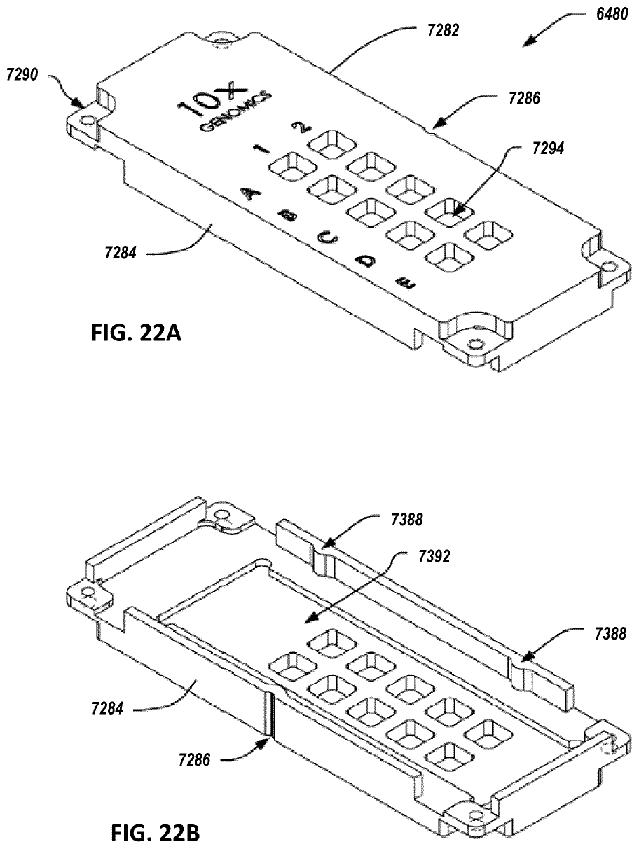

Referring to FIGS. 22A and 22B, the top member 6480 can include a body 7282 with walls 7284. In some embodiments, one side of the body 7282 and/or the wall 7284 can include a marker 7286. Marker 7286 can aid in coupling the bottom member 6460 and the top member 6480 in a certain orientation. For example, in some embodiments, the bottom member 6460 and the top member 6480 may only be able to couple together in a single orientation. In some embodiments, the marker 7286 can be a line, groove, indent, protrusion, symbol, color, etc. to distinguish one side of the body 7282 from another side of the body 7282.

In some embodiments, the walls 7284 can be configured to engage with the bottom member 6460. For example, in some embodiments, the walls 7284 can include one or more protrusions 7388. In some embodiments, a first wall 7284 can include a single protrusion 7388, while a second wall 7284 can includes multiple protrusions 7388 (e.g., two protrusions). Such a configuration can provide single direction coupling of the bottom member 6460 and the top member 6480 for ease of use.

The body 7282 can further include an engagement mechanism for coupling the top member 6480 to the bottom member 6460. In some embodiments, the engagement mechanism includes screws 6496. Accordingly, the body 7282 can include one or more threaded apertures 7290 configured to receive the screw 6496. The body 7282 can be configured such that when the apertures 7290 receive the screws 6496, the heads of the screws 6496 are flush or lower than an upper face of the body 7282. Accordingly, the top member 6480 can be configured such that a top plate of a heating device can abut the top member 6480, providing heating of the substrate holder 6150.

In some embodiments, the body 7282 can include a recess 7392 on the underside of the body 7282. In some embodiments, the recess 7392 can receive the gasket 6454. In some embodiments, the recess 7392 can removably couple the gasket 6454. In some embodiments, the body 7282 can include the gasket 6454, such that the gasket 6454 is integrated with the body 7282.

In some embodiments, the body 7282 can include a plurality of apertures 7294. In some embodiments, the plurality of apertures 7294 can be configured to enable reagents to be added to the substrate on the slide 6452. In some embodiments, the plurality of apertures 7294 can be configured to be aligned with the plurality of apertures 7156 of the gasket 6454. In some embodiments, the plurality of apertures 7294 can be configured with a format and spacing to enable use with a multichannel pipette. In some embodiments, the plurality of apertures 7294 can be located on only a portion of the body 7282. In some embodiments, the body 7282 can include labels or markings adjacent to the plurality of apertures 7294.

While the substrate holder 6150 is described as including multiple pieces (e.g., the bottom member 6460, the top member 6480, the slide 6452, the gasket 6454, etc.), components of the substrate holder 6150 can be integrated with one another. For example, in some embodiments, the gasket 6454 can be integrated with the top member 6480. As another example, the top member 6480 and the bottom member 6460 can be a single piece that is configured to receive the slide 6452.

In some embodiments, the substrate holder 6150 can be made of a material that is reusable. For example, in some embodiments, the substrate holder 6150 can be washed and sanitized for reuse. Optionally, the gasket 6454 can be reusable or replaceable in such an embodiment. In some embodiments, the substrate holder 6150 can be made for single use and can be disposable.

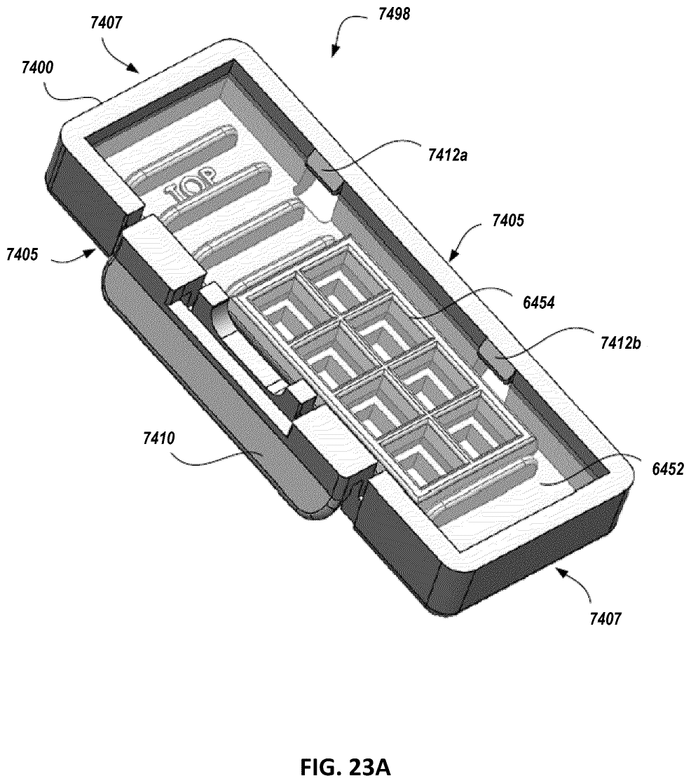

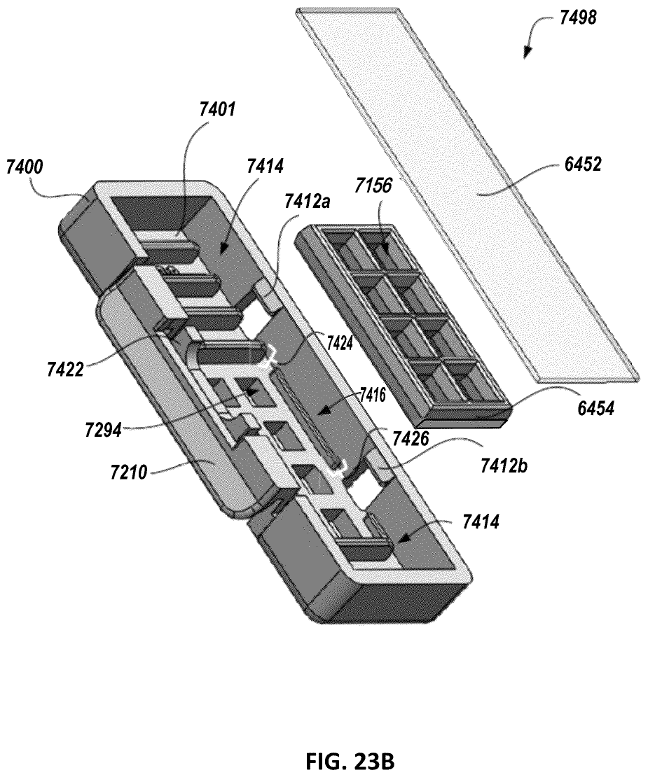

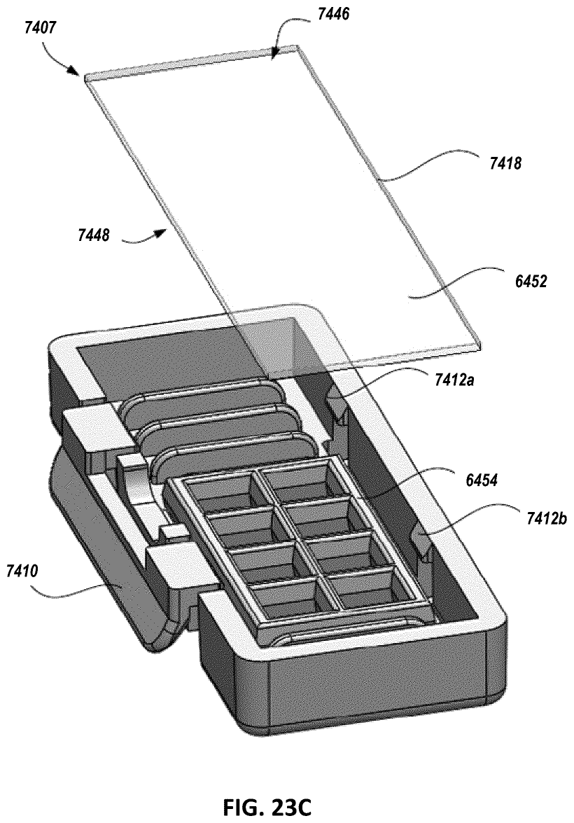

Referring to FIGS. 23-25, another example substrate cassette is described. An example device 7498 can be used to replace the substrate cassettes 4010, 4810 described herein. The device 7498 can include a substrate holder 7400, a gasket 6454, and a substrate, such as a glass slide 6452. The slide 6452 includes a first surface and a second surface. In some embodiments, the substrate holder 7400 is configured to receive the slide 6452. In some embodiments, the substrate holder 7400 includes an attachment mechanism to hold the slide 6452 to the substrate holder 7400. The second surface of the slide 6452 can provide a substrate for receiving a sample. In some embodiments, the substrate holder 7400 is plastic component (e.g., injection molded plastic component). In some embodiments, the gasket 6454 is configured to be positioned in between the substrate holder 7400 and the slide 6452. In some embodiments, the device 7498 (or any one of its components) can be a single-use device (or component). In some embodiments, the device 7498 is entirely disposable or at least partially composed of disposable components.

FIG. 23A shows the device 7498 in an assembled state. In particular, FIG. 23A shows the substrate holder 7400 receiving the gasket 6454 and the slide 6452. The substrate holder 7400 has longitudinal sides 7405 and latitudinal sides 7407. The substrate holder 7400 can include one or more fasteners, such as a side mounted press latch 7410 for snap engagement. Any type of fastener that allows releasable engagement can be used, such as, for example, screws and press fit type connectors. The press latch 7410 can be mounted in a longitudinal side 7505 of the substrate holder 7400, as shown in FIGS. 23A-23C. Alternatively, in some embodiments, the press latch 7410 can be mounted in a latitudinal side 7407 of the substrate holder 7400. In some embodiments, the substrate holder 7400 can include two, three, or four press latches. The press latch 7410 can be configured to engage the slide 6452. In some embodiments, the press latch 7410 can be a lever, a clip, or a clamp. In some embodiments, the press latch 7410 can further include one or more springs.

The substrate holder 7400 can further include one or more engagement features, such as a first tab 7412a and a second tab 7412b. The first and second tabs 7412a and 7412b, respectively, can protrude from a longitudinal side 7505 of the substrate holder 7400 that opposes the longitudinal side having the press latch 7410. In some embodiments, the substrate holder 7400 includes three, four, five, six, seven, eight, nine, ten or more tabs. In some embodiments, the tabs protrude from a longitudinal side 7505 or a latitudinal side 7407 of the substrate holder 7400. The first and second tabs 7412a and 7412b can be configured to engage the slide 6452. In some embodiments, the first and second tabs 7412a and 7412b are rigid and do not flex when engaging the slide 6452. In some embodiments, the tabs 7412a and 7412b can be flexible.

Referring to FIG. 23B, the substrate holder 7400 includes a bottom surface 7401. The bottom surface 7401 includes a plurality of latitudinal ribs 7414 and longitudinal ribs 7416 configured to support the slide 6452 and the gasket 6454 when the device 7498 is assembled. The bottom surface 7401 further defines a plurality of apertures 7294 that are configured to align with the plurality of apertures 7156 defined by the gasket 6454, when the device 7498 is assembled. The substrate holder 7400 further includes a c-shaped tab 7422 shaped to provide the user with an ergonomic grip surface to help facilitate engagement with the press latch 7410.

Referring to FIG. 23C, the slide 6452 includes a first surface 7446, a second surface 7448, and a side edge 7418. When inserting the slide 6452 into the substrate holder 7400, the side edge 7418 can be inserted first such that first and second tabs 7412a and 7412b engage the side edge 7418 as the slide 6452 rests on the gasket 6454 an on the plurality of latitudinal ribs 7414. In some embodiments, the side 7418 can measure about 6 inches. In some embodiments, the sides shorter than the side 7418 can measure about 1 inch. In some embodiments, the slide 6452 can measure about 75 millimeters (mm) by 25 mm. In some embodiments, the slide 6452 can measure about 75 millimeters (mm) by 50 mm. In some embodiments, the slide 6452 can measure about 48 millimeters (mm) by 28 mm. In some embodiments, the slide 6452 can measure about 46 millimeters (mm) by 27 mm. In some embodiments, the slide 6452 is a glass slide.

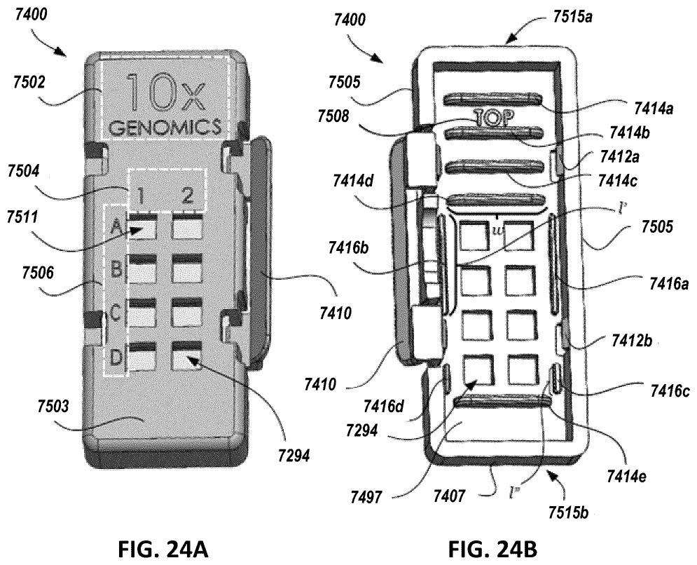

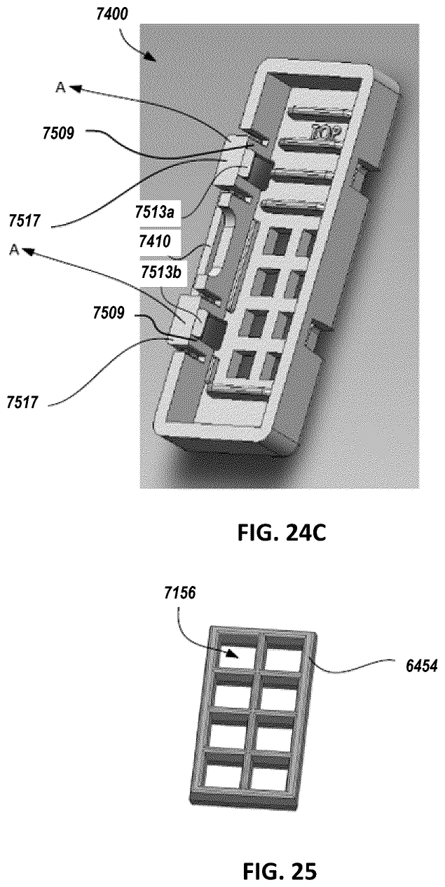

Referring to FIG. 24A, the substrate holder 7400 includes a top surface 7503. The top surface 7503 defines the plurality of apertures 7294. Furthermore, the top surface 7503 includes a logo 7502, a first identifier 7504, and a second identifier 7506. The first identifier 7504 can identify the columns of the plurality of apertures 7294. The second identifier 7506 can identify the rows of the plurality of apertures 7294. In some embodiments, the first identifier 7504 and the second identifier 7506 are letters or numbers. In some embodiments, the first identifier 7504 and the second identifier 7506 can aid in aligning and/or inserting the slide 6452 into the substrate holder 7400 in a certain orientation. In some embodiments, the slide 6452 may include samples (e.g., biological material samples) on a portion of its surface that align with one or more of the plurality of apertures 7294. In some embodiments, the samples (e.g., biological material samples) may be identified in the same manner as the corresponding aperture of the plurality of apertures 7294. For example, in some embodiments, the slide 6452 may include a sample named “A1” that corresponds with the aperture 7511 labeled as “A1” by the first identifier 7504 and the second identifier 7506 in FIG. 24A. As such, in some embodiments, the first identifier 7504 and the second identifier 7506 guide a user to correctly place the slide 6452 into the substrate holder 7400. In some embodiments, the first identifier 7504 and the second identifier 7506 can be a line, groove, indent, protrusion, symbol, color, etc. to distinguish the rows and columns of the plurality of apertures 7294.

Referring to FIG. 24B, the substrate holder 7400 includes a first latitudinal rib 7414a, a second latitudinal rib 7414b, a third latitudinal rib 7414c, a fourth latitudinal rib 7414d, and a fifth latitudinal rib 7414e having a width w. The plurality of latitudinal ribs 7414 is configured to support a slide 6452. The first, second, third, fourth, and fifth latitudinal ribs 7414a, 7414b, 7414c, 7414d, and 7414e can have an equal width w, as shown in FIG. 24B. In some embodiments, the widths of the plurality of latitudinal ribs can vary. The first, second, and third latitudinal ribs 7414a, 7414b, and 7414c, respectively, extend perpendicular from the bottom surface 7401 near a first end 7515a of the substrate holder 7400. The fifth latitudinal rib 7414e extends substantially perpendicular from the bottom surface 7401 near a second end 7515b of the substrate holder 7400. In some embodiments, the substrate holder 7400 may include 6, 7, 8, 9, 10, 15, 20 or more latitudinal ribs.

The substrate holder 7400 further includes a first longitudinal rib 7416a, a second longitudinal rib 7416b, a third longitudinal rib 7416c, and a fourth longitudinal rib 7416d that extend substantially perpendicular from the bottom surface 7401. The plurality of longitudinal ribs 7416 is configured to provide longitudinal support to the gasket 6454. For example, in some embodiments, the plurality of longitudinal ribs 7416 abuts the longitudinal sides of the gasket 6454. Furthermore, together with the fourth and fifth latitudinal ribs 7414c and 7414e, the plurality of longitudinal ribs frame an area of the bottom surface 7401 (e.g., gasket area) that is sufficiently sized and configured to receive the gasket 6454. The first longitudinal rib 7416a, second longitudinal rib 7416b, third longitudinal rib 7416c, and fourth longitudinal rib 7416d can be disposed parallel to the longitudinal sides 7505 along the side edges. The first longitudinal rib 7416a, second longitudinal rib 7416b, third longitudinal rib 7416c, and fourth longitudinal rib 7416d have a second height 7426, as shown in FIG. 24B. The first, second, third, fourth, and fifth latitudinal ribs 7414a, 7414b, 7414c, 7414d, and 7414e have a first height 7424, as shown in FIG. 32B. The first height 7424 can be greater than the second height 7426, as shown in FIGS. 32-33. In some embodiments, the first height 7424 is equal to the second height 7426 (see FIG. 23B). In some embodiments, the first height 7424 is less than the second height 7426. The first and second longitudinal ribs 7416a and 7416b have a length l′. The third and fourth longitudinal ribs 7416c and 7416d have a length/“. The length l′is can be greater than the length/”, as shown in FIG. 23B. In some embodiments, the length l′is equal to the length/“. In some embodiments, the length l′is less than the length/”. In some embodiments, the substrate holder 7400 may include 5, 6, 7, 8, 9, 10, 15, 20 or more longitudinal ribs.

The substrate holder 7400 further includes a third identifier 7508 that aids a user in positioning and/or orienting the substrate holder 7400. For example, in some embodiments, the third identifier 7508 aids in identifying the first end 7515a or aids in distinguishing the first end 7515a from the second end 7515b. Still yet in further embodiments, the third identifier can be a line, groove, indent, protrusion, symbol, color, etc. that aids a user in correctly positioning slide 6452 into the substrate holder 7400.

Referring to FIG. 24C, the substrate holder includes a first flexible tab 7513a and a second flexible tab 7513b. The first and second flexible tabs 7513a and 7513b, respectively, can protrude from top portions 7517 of the press latch 7410 that oppose the longitudinal side 7505 having the first and second tabs 7412a and 7412b. The first and second flexible tabs 7513a and 7513b can protrude from an interior surface 75109 of the top portions 7517 of the press latch 7410. To utilize the substrate holder 7400, a user can grip the substrate holder 7400 in one hand with the bottom surface 7401 face up (i.e., facing the user). Next, the user can place the gasket 6454 over the plurality of apertures 7294. The user can depress the press latch 7410 with one hand, this flexes the first and second flexible tabs 7513a and 7513b on one longitudinal side 7505 open, in the direction of arrows A. As such, the user can load the slide 6452 into the first and second tabs 7412a and 7412b first, and subsequently hinge slide 6452 into opposite longitudinal side (i.e., the side having the first and second flexible tabs 7513a and 7513b). Lastly, the user can release the press latch 7410 so the slide 6452 can snap into the first and second flexible tabs 7513a and 7513b.

Referring to FIG. 25, the gasket 6454 includes a plurality of apertures 7156. In some embodiments, the gasket 6454 includes eight apertures. In some embodiments, the gasket 6454 includes sixteen apertures. In some embodiments, the gasket 6454 includes 24 apertures. In some embodiments, the gasket 6454 includes 96 apertures. In some embodiments, the gasket 6454 is made from a material that can withstand temperatures up to about 60 degrees Celsius. In some embodiments, the gasket 6454 is made from a heat-resistant material. In some embodiments, the gasket 6454 is made from a flexible or pliable material. Non-limiting examples of flexible or pliable materials include rubber, silicone, and polyurethane. It is contemplated that the number of apertures is the same as the number of sample arrays on a substrate.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363 (6434):1463-1467, 2019; Lee et al., Nat. Protoc. 10 (3): 442-458, 2015; Trejo et al., PLOS ONE 14 (2):e0212031, 2019; Chen et al., Science 348 (6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I) (b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up-and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21;45 (14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1-38. (canceled)

39. An electrophoretic system for migrating analytes from a biological sample, the electrophoretic system comprising:
a substrate including a substrate region configured to place a capture probe thereon, the substrate region configured to receive the biological sample containing the analytes, the substrate configured to be usable as an anode;
a cathode spaced apart from the substrate;
a buffer chamber between the substrate and the cathode and configured to contain a buffer; and
a controller configured to generate an electric field between the substrate and the cathode such that the analytes in the biological sample migrate toward the capture probe on the substrate.

40. The electrophoretic system of claim 39, wherein the substrate includes a conductive material, or the substrate is coated with a conductive material.

41. The electrophoretic system of claim 40, wherein the conductive material includes at least one of tin oxide (TO), indium tin oxide (ITO), a transparent conductive oxide (TCO), aluminum doped zinc oxide (AZO), or fluorine doped tin oxide (FTO).

42. The electrophoretic system of claim 39, wherein the substrate is a glass slide.

43. The electrophoretic system of claim 39, wherein the substrate comprises an array of substrate regions configured to place capture probes thereon.

44. The electrophoretic system of claim 39, wherein the substrate comprises an array of features, where each feature of the array of features is associated with a unique spatial location on the array.

45. The electrophoretic system of claim 39, wherein the capture probe is immobilized on the substrate region.

46. The electrophoretic system of claim 39, wherein the capture probe comprises a spatial barcode, a unique molecular identifier, and a capture domain.

47. The electrophoretic system of claim 39, further comprising a spacer disposed between the substrate and the cathode to define the buffer chamber.

48. The electrophoretic system of claim 39, further comprising the buffer in the buffer chamber, optionally wherein the buffer includes a permeabilization reagent.

49. The electrophoretic system of claim 48, wherein the substrate, the cathode, or both are at least partially immersed in the buffer.

50. The electrophoretic system of claim 39, further comprising:
a power supply; and
electrical wires connecting the power supply to the substrate and the cathode.

51. The electrophoretic system of claim 39, further comprising:
a substrate cassette configured to hold the substrate and including a plurality of apertures configured to define a plurality of buffer chambers on the substrate.

52. An electrophoretic system comprising:
a substrate including a plurality of substrate regions comprising capture probes and one or more biological samples containing analytes;
a substrate cassette configured to hold the substrate and including a plurality of apertures corresponding to the plurality of substrate regions of the substrate, the plurality of apertures configured to define a plurality of buffer chambers on the plurality of substrate regions of the substrate;
a cathode assembly including a plurality of electrode plates, the plurality of electrode plates configured to position within the plurality of buffer chambers of the substrate cassette;
a controller configured to generate electric fields between the plurality of substrate regions and the plurality of electrode plates such that the analytes in the one or more biological samples migrate toward the capture probes on the substrate; and optionally
a power supply and electrical wires connecting the power supply to the cathode assembly and each of the plurality of substrate regions of the substrate.

53. The electrophoretic system of claim 52, wherein the one or more biological samples are placed in contact with the capture probes on the plurality of substrate regions.

54. The electrophoretic system of claim 52, wherein the plurality of substrate regions includes a plurality of wells recessed on the substrate.

55. The electrophoretic system of claim 52, wherein the substrate cassette comprises:
a substrate holder including a substrate mount for securing the substrate; and
a gasket including a plurality of gasket apertures configured to align with the plurality of substrate regions when the substrate is secured by the substrate holder, wherein the plurality of apertures includes the plurality of gasket apertures.

56. The electrophoretic system of claim 55, wherein the substrate holder comprises:
a plurality of holder apertures configured to align with the plurality of gasket apertures when the substrate is secured by the substrate holder, wherein the plurality of apertures includes the plurality of gasket apertures and the plurality of holder apertures.

57. The electrophoretic system of claim 52, further comprising:
a substrate cover configured to be arranged on the substrate cassette and including a plurality of cover apertures configured to align with the plurality of apertures of the substrate cassette,
wherein the cathode assembly is mounted to the substrate cover and the plurality of electrode plates of the cathode assembly is configured to extend through the plurality of cover apertures into the plurality of apertures of the substrate.

58. The electrophoretic system of claim 52, wherein the substrate is coated with a conductive material, optionally wherein the conductive material comprises at least one of:
tin oxide (TO), indium tin oxide (ITO), a transparent conductive oxide (TCO), aluminum doped zinc oxide (AZO), and fluorine doped tin oxide (FTO).
